(19) United States
(12) Patent Application Publication
(10) Pub. No.: US 2023/0295271 A1
(43) Pub. Date: Sep. 21, 2023

(54) METHODS AND COMPOSITIONS FOR IN VITRO AFFINITY MATURATION OF MONOCLONAL ANTIBODIES

(71) Applicant: University of Southern California, Los Angeles, CA (US)

(72) Inventors: Myron F. Goodman, Los Angeles, CA (US); Michael Lieber, Los Angeles, CA (US); Phuong Pham, Los Angeles, CA (US)

(73) Assignee: University of Southern California, Los Angeles, CA (US)

(21) Appl. No.: 17/928,690

(22) PCT Filed: Jun. 2, 2021
(86) PCT No.: PCT/US2021/035528

§ 371 Date: Nov. 30, 2022

Related U.S. Application Data
(60) Provisional application No. 63/033,429, filed on Jun. 2, 2020.

(51) Int. Cl. C07K 16/00; C12N 15/10; C07K 16/28; C07K 16/40

(52) U.S. Cl. CPC C07K 16/005; C12N 15/1093; C07K 16/2869; C07K 16/40; C07K 2317/622

(57) ABSTRACT

Methods and systems are provided to induce hypermutation, produce phage libraries displaying mutagenized immunoglobulin variable regions, and conduct affinity maturation based on the use of activation-induced deoxycytidine deaminase (AID) and a low fidelity DNA polymerase eta (pol η). High affinity monoclonal antibodies or antigen-binding fragments thereof produced by these methods are also demonstrated.

### CROSS-REFERENCE TO RELATED APPLICATIONS

This application includes a claim of priority under 35 U.S.C. § 119(e) to U.S. provisional patent application No. 63/033,429, filed Jun. 2, 2020, the entirety of which is hereby incorporated by reference.

### STATEMENT REGARDING FEDERALLY SPONSORED RESEARCH OR DEVELOPMENT

This invention was made with Government support under Grant No. ES028343 awarded by National Institutes of Health. The government has certain rights in the invention.

### REFERENCE TO SEQUENCE LISTING

The Sequence Listing submitted Jun. 2, 2021 as a text file named “SequenceListing-065715-000099WO00_ST25” created on Jun. 2, 2021 and having a size of 33,400 bytes, is hereby incorporated by reference.

### FIELD OF INVENTION

This invention relates to methods and systems for generating monoclonal antibodies.

### BACKGROUND

Human antibodies (Abs) are made in immune B-cells, first as low-affinity Abs that recognize and bind weakly to invading antigen (Ag) molecules via a process called VDJ recombination, and second as high affinity Abs that bind tightly to an Ag, via somatic hypermutation (SHM) of the Ag-binding, immunoglobulin variable domain (IgV). That is, the variable domains are assembled by a process called V-(D)-J rearrangement and can then be subjected to SHM which, after exposure to antigen and selection, allow affinity maturation for a particular antigen. When a B-cell has “identified” a sufficiently tight binding Ab, it then clonally expands the B-cell during which affinity maturation occurs to produce high-affinity Abs.

Specifically, as a major component of the process of affinity maturation, somatic hypermutation (SHM) involves a programmed process of mutation affecting the variable regions of immunoglobulin genes. Unlike germline mutation, SHM affects only an organism's individual immune cells. When a B cell recognizes an antigen, it is stimulated to divide (or proliferate). During proliferation, the B-cell receptor locus undergoes an extremely high rate of somatic mutation (e.g., orders of magnitude greater than the normal rate of mutation across the genome) mainly in the form of single-base substitutions, with insertions and deletions being less common. These mutations occur mostly at “hotspots” in the DNA, which are concentrated in hypervariable regions, which correspond to the complementarity-determining regions, i.e., the sites involved in antigen recognition on the immunoglobulin. The “hotspots” of somatic hypermutation vary depending on the base that is being mutated, and the overall result of the hypermutation process is achieved by a balance between error-prone and high fidelity repair. This directed hypermutation allows for the selection of B cells that express immunoglobulin receptors possessing an enhanced ability to recognize and bind a specific foreign antigen.

Further by a mechanism called class-switch recombination (CSR) binding, immunoglobulin class switching (also known as isotype switching or isotypic commutation) occurs after activation of a mature B cell via its membrane-bound antibody molecule (or B cell receptor) to generate the different classes of antibody (i.e., with the same antigen specificity—variable domains as the original antibody generated in the immature B cell, but possessing different constant domains in the heavy chain). Naïve mature B cells produce both IgM and IgD, which are the first two heavy chain segments in the immunoglobulin locus. After activation by antigen, these B cells proliferate; and if these activated B cells encounter specific signaling molecules, they undergo antibody class switching to produce IgG, IgA or IgE antibodies. During CSR, the constant-region portion of the antibody heavy chain is changed, but the variable region of the heavy chain stays the same, therefore class switching does not affect antigen specificity. This allows different daughter cells from the same activated B cell to produce antibodies of different isotypes or subtypes (e.g. IgG1, IgG2 etc.), which can interact with different effector molecules.

Typically, monoclonal Abs are made commercially by injecting an animal, rabbit, mouse, llama, with an Ag and then let the animal produce the Ab. This is a lengthy and costly process that needs to be repeated for each Ag.

Therefore, it is an objective of the present invention to provide alternative methods to produce antibodies. It is another objective of the present invention to provide systems and methods for generating IgV mutations in vitro and performing affinity maturation selection to produce high-affinity monoclonal antibodies.

### SUMMARY OF THE INVENTION

Various embodiments provide in vitro methods, which include providing a library of phage or phagemid clones; diversifying the library of phage or phagemid clones by contacting the library of phage or phagemid clones with activation-induced deoxycytidine deaminase (AID); further diversifying the library of phage or phagemid clones by contacting the library of phage or phagemid clones with DNA polymerase eta (Pol η); and transfecting the diversified library into bacteria; and generating a phage library.

Further embodiments of the methods include panning the phage or phagemid library against an antigen. In some aspects, the diversification, the further diversification, the transfecting, the generating, and the panning are repeated one time, two times, three times, four times, five times, six times, seven times, eight times, or more in the methods. In some aspects, the diversification, the further diversification, the transfecting, the generating, and the panning are performed for 2-4 times. In some aspects, the diversification, the further diversification, the transfecting, the generating, and the panning are performed for 3-5 times. In some aspects, the diversification, the further diversification, the transfecting, the generating, and the panning are performed for 4-6 times. In some aspects, the diversification, the further diversification, the transfecting, the generating, and the panning are performed for 5-7 times. In some aspects, the diversification, the further diversification, the transfecting, the generating, and the panning are performed for 6-8 times. In some aspects, the diversification, the further diversification, the transfecting, the generating, and the panning are performed for 7-9 times. In some aspects, the diversification, the further diversification, the transfecting, the generating, and the panning are performed for 8-10 times.

In some embodiments, the library of phage clones is made by a method, comprising providing a library of naïve IgV genes; diversifying the library of naïve IgV genes by contacting the library of naïve IgV genes with activation-induced deoxycytidine deaminase (AID); further diversifying the library of naïve IgV genes by contacting the library of naïve IgV genes with DNA polymerase eta (Pol η); generating a phage library; and (optionally) further comprising panning the phage library against at least one antigen to generate the library of phage clones.

In various embodiments, inducing mutagenesis or hypermutation comprises formation of a single stranded DNA cassette to generate gapped DNA, and contacting the gapped DNA with Pol η to initiate low-fidelity synthesis to fill the gap. In various aspects, a DNA substrate or the gapped DNA is first treated with AID, before proceeding with the low-fidelity synthesis. This generally provides a higher count of mutations. In other aspects, the gapped DNA can undergo the Pol η-mediated low-fidelity synthesis first, followed by treatment of AID to deaminate. In yet other aspects, a DNA substrate is first treated with AID, then undergoes Pol η-mediated low-fidelity synthesis, and subsequently followed by another AID treatment.

Methods are provided for preparation of monoclonal antibodies (MAb) or antigen-binding fragments thereof, which include inducing mutagenesis in phage or phagemid libraries displaying one or more IgVs (e.g., scFv, VHH nanobody), transfecting the mutagenized phage or phagemid libraries into bacteria, and panning the phage or phagemid libraries against an antigen, in an affinity maturation process.

In some embodiments, AID and pol η-mutagenized Ab libraries are used in phage bio-panning to screen and select for initial Abs which bind to target antigens. In some embodiments, the initial Ab clones are treated with AID and pol η, followed by successive rounds of bio-panning, to perform Ab maturation in a test tube (e.g., without B cells, T cells or lymphocytes; without mammalian cells).

High affinity antibodies and antigen-binding fragments produced by these methods are also provided, including those against glucagon-like peptide-1 receptor, AID, artemin, or fatty acid amide hydrolase.

### BRIEF DESCRIPTION OF FIGURES

FIG. 1 shows that AID is required for somatic hypermutation (SHM) and Class Switch Recombination (CSR) of Ig genes. Low affinity IgM-producing IgV_{H} genes undergo SHM and CSR which require C→U deamination by AID at variable (V) and switch (S) regions, respectively. IgV promoter (P) is needed for AID access to V regions, whereas germline transcription from the I promoters is required for AID targeting to the donor switch (Sμ) and a downstream acceptor S. Error-prone processing of G:U mispairs involving a polymerase η introduces a huge number of mutations (˜10⁻⁴ to 10⁻³ per base per generation) preferentially at WRC (AID hot motifs) and WA (pol η hot motifs) in the antigen-binding V region. CSR combines the V exon with one of the appropriate downstream constant C regions γ3, ε or α, converting IgM or IgD to other isotypes IgG3, IgE, or IgA. The graph on top left side shows a typical SHM profile. The inset on the top right side illustrates a typical human Ig molecule composed of two heavy and two light chains with Ag-interacting variable region (V) and constant region (C).

FIGS. 2A and 2B depict antibody affinity maturation workflow in a test tube. FIG. 2A depicts that antigen-binding single chain variable fragments (scFv) of Abs are expressed as fusion proteins on a minor coat protein, pIII, of filamentous phage. FIG. 2B depicts a schematic diagram showing the steps involved in antibody affinity maturation in a test tube. V gene hypermutation is carried out by (1) treatment of gapped scFv phagemid (wherein the scFv region is exposed as ssDNA, as there exists a gap in the other strand) with human AID, in that AID targets ssDNA V region at WRC hot motifs to generate mutations at G/C sites (e.g., up to 60% of gap DNA can be mutated in a single reaction); followed by (2) treatment with error-prone gap-filling synthesis, Pol η, in that Pol η generates additional mutations at A/T sites (e.g., (mutation rate is about 1×10⁻²). Then (3) hypermutated scFv phages are packaged in E. coli, e.g., transformation into E. coli to generate scFv phage display library, in that mutation patterns in V genes are similar to SHM mutation in B-cells. Then (4) the resulting hypermutated phage scFv clones are subjected to phage bio-panning against a chosen target antigen to select for bound clones; and (5) selected antigen-specific scFv phages are subjected to additional rounds of in vitro affinity maturation procedure to obtain the desired high (and specific) affinity scFv clones.

FIG. 3 shows that purified human AID and pol η catalyzed highly diversified mutations on human IgHV3-23*01 substrate in a test tube. (Gene IGHV3-23 encodes immunoglobulin heavy variable 3-23, which belongs to a cluster of approximately 40 functional variable (V) genes in the Ig heavy chain locus on chromosome 14; *01 refers to V gene allele name.) FIG. 3 depicts that a gapped DNA substrate (with lacZ-IgVH3-23*01 exposed as ssDNA) was incubated with AID, followed by error-prone gap-filling synthesis by pol η. DNA products were used to transform E. coli and plated on α-complementation host cells. AID-catalyzed C deamination (C→U) are copied giving rise to C→T mutations. Pol η makes mutations at other sites during gap-filling synthesis. Mutations in lacZ (in the lacZ-IgHV3-23*01 region) change M13 phage (dark blue color) to mutant M13 phage (light blue or white color). The mutation result by AID and pol η is described in Example 1.I.

FIG. 4 shows a “double gap” procedure to construct partially gapped scFv substrates on both DNA strands. Phagemid dsDNA without scFv (pADL20) is linearized with BglI restriction enzyme; and the DNA containing a scFv insert (pADL20-scFv) is linearized with PvuI. The linearized DNAs are denatured in water at high temperature, quickly mixed and cooled down in the presence of salt to form circular gapped constructs with the scFv region exposed as ssDNA (sense or anti-sense strand). A significantly higher number of transformants following denaturing/annealing procedure (1724 vs. 53 and 9), indicating of an efficient gap molecule formation. That is, the number of transformants (˜10 pg DNA) for linearized pADL20 is 9, for linearized pADL20-scFv is 53, and for circular “gap” pADL20-scFv is 1724.

FIG. 5 shows an exemplary procedure to hypermutate a scFv-phages by AID and pol η. Circular gapped scFv-phage substrates were subjected to AID deamination by incubation with various amounts of AID and incubation times (from 30 seconds to 30 minutes), followed by error-prone gap filling synthesis by pol η in the presence of dNTP. Following AID and pol η treatment, DNAs are pooled and used for E. coli transformation to make a mutagenized scFv phage display library. Heavy (VH) and light (VL) chain identities and numbers of mutations caused by AID and pol η mutations are shown in Table 1 for each sequenced scFv clone.

FIGS. 6A and 6B depict VH and VL sequences, respectively, of a representative human scFv clone (F3) from the human mutagenized scFv library. FIG. 6A depicts alignment of F3 VH domain to germ line IGHV6-1*01 genes showed 4 mutations, all C to T (square-boxed highlights) consistent with the AID deamination signature in vitro. VH sequence of F3 clone is also provided in SEQ ID NO:109. FIG. 6B depicts that the light chain of F3 contains 48 mutations with a significant number of C to T and G to A mutations (19 square-boxed highlights), indicating that mutations in the VL chain arose from an Ab produced in B-cells during secondary immune responses and additionally during AID and Pol η diversification in vitro. VL sequence of F3 clone is also provided in SEQ ID NO:110. scFv sequence analysis was performed using IgBLAST tool.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed, Revised, J. Wiley & Sons (New York, NY 2006), and Sambrook and Russel, Molecular Cloning: A Laboratory Manual 4^{th} ed., Cold Spring Harbor Laboratory Press (Cold Spring Harbor, N Y 2012), provide one skilled in the art with a general guide to many of the terms used in the present application.

The term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

The phrase “phagemid” refers to a DNA-based cloning vector, which carries an origin of replication derived from bacteriophage in addition to the origin of plasmid replication. Typically, a plasmid replication origin allows isolation of double stranded (supercoiled) plasmid from the cytoplasm of cells after disrupting the cells; and a replication origin usually from a single stranded phage such as f1, fd or M13 allows the plasmid to enter a single strand replication mode in which only one of the (Watson/Crick) strands is packaged into the virus particle when helper phage is added to the cell carrying the phagemid. Phagemids usually encode no or only one kind of coat proteins. For example, libraries constructed in phagemid vectors can be fusions (of protein of interest) to filamentous phage protein III (pIII), yielding primarily single copy (monovalent) display of antibody fragments. A true phage vector can create a multivalently displayed antibody fragments. Phagemids and phage can be used in the technique of “phage display”.

The phrase “phage display” refers to a high-throughput screening technique of protein-protein, protein-peptide, and protein-DNA interactions. Specifically, a gene encoding a protein of interest is inserted into a phage coat protein gene, thereby causing the phage to “display” the protein on its outside while containing the gene for the protein on its inside (i.e., resulting in a connection between genotype and phenotype), and these displaying phages can then be screened against other proteins, peptides or DNA sequences in order to detect interaction between the displayed protein and those other molecules. Usually, large libraries of proteins can be screened and amplified in this in vitro selection process. Exemplary bacteriophages used in phage display include M13 phage, fd filamentous phage, T4, T7, and λ phage.

“Phages” or “bacteriophages” are used interchangeably to refer to viruses that infect and replicate within bacteria and/or archaea.

“Hypermutation” or “hypermutate” can refer to induced mutagenesis/mutations with respect to a gene or a collection of loci of interest that are significantly higher in rate or frequency, compared to natural mutations in the gene or loci of interest or to the normal rate/frequency of mutation across the genome. In some embodiments, hypermutations are orders of magnitude (e.g., at least 50-fold, 100-fold, 10³-fold, or 10⁴-fold) higher than natural mutations or normal rate of mutations across the genome. In some embodiments, hypermutations induced by Pol η generates mutations at A/T sites at a rate of about 1×10⁻².

The term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g., papain cleavage) or via chemical cleavage of intact antibodies. An antibody can be a chimeric, humanized or human antibody. An antibody can be an IgG1, IgG2, IgG3 or IgG4 antibody. In some aspects, an antibody herein has an Fc region that has been modified to alter at least one of effector function, half-life, proteolysis, or glycosylation.

The term “antibody fragment,” refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and CH1 domains; (ii) the Fab′ fragment, which is a Fab fragment having one or more cysteine residues at the C-terminus of the CH1 domain; (iii) the Fd fragment having V_{H} and CH1 domains; (iv) the Fd′ fragment having V_{H} and CH1 domains and one or more cysteine residues at the C-terminus of the CH1 domain; (v) the Fv fragment having the V_{L} and V_{H} domains of a single arm of an antibody; (vi) the dAb fragment which consists of a V_{H} domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv); (x) “diabodie” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}-CH1-V_{H}-CH1) which, together with complementary light chain polypeptides, form a pair of antigen binding regions. An antibody or antibody fragment can be scFvs, camelbodies, nanobodies, IgNAR (single-chain antibodies derived from sharks) and Fab, Fab′ or F(ab′)₂ fragment.

Antibody molecules or fragments may be of any Ig (immunoglobulin) isotype, such as IgG, IgM or IgA, etc.

“Selectively binds” or “specifically binds” refers to the ability of an antibody or antibody fragment thereof described herein to bind to a target, such as a molecule present on the cell-surface, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

“Biopanning” generally refers to using an affinity selection technique to select for peptides that bind to a given target. Biopanning typically involves preparing phage display libraries (e.g., inserting desired gene segments into phage genome so it expresses with pIII or pVIII of phage M13), contacting the phage library to a desired target for binding interactions (also known as “panning”), washing away unbound phages, and eluting the bound phages for collection. The collected phages can infect bacteria once again to produce phage libraries.

The term “repertoire” refers to an assortment of immunoglobulins or fragments thereof. It can preferably contain a diversity of at least about 10², 10³, or 10⁴ and as high as 10¹³, or any range or value therein, of different species. A “library” can preferably contain a diversity of at least about 10², 10³, or 10⁴, typically >10¹⁰, or as high as 10¹³, or any range or value therein, of different species (e.g., having high complexity).

SHM is a complex process, see the end of the Examples for a brief description of SHM and CSR. To mimic the natural SHM mutations occurring in activated B-cells and circumvent the lengthy and costly process associated with antibody production in animals, the Inventors have reconstituted SHM in a test tube by combining activities of purified human AID and Pol η to recreate a wide range of IgV mutations which recapitulate SHM mutational diversity in B-cells. Our technology allows the production of high-affinity monoclonal Abs in a test tube. The Inventors have exposed naïve IgV genes obtained from human tonsil, and separately from llama lymphocytes, to AID and pol η. The Inventors then used the mutagenized IgV genes to generate Ab libraries, in the form of single-chain variable Abs (scFv for human and VHH for Llama) expressed on the surface of filamentous bacteriophages. AID and pol η-mutagenized Ab libraries are used in phage bio-panning to screen and select for initial Abs which bind to target antigens (Ags). The power of our technology resides in the next series of steps, in which the Inventors then perform Ab maturation in the test tube by treating the initial Ab clones with AID and pol η followed by successive rounds of bio-panning, e.g., one round, two rounds, three rounds, four rounds, five round, six round, seven rounds, or eight rounds, or more; in some instances, 2-4 rounds, 3-5 rounds, 4-6 rounds, 5-7 rounds, 6-8 rounds, 7-9 rounds, or 8-10 rounds. The kernel of our technology is successfully performing the essential enzymatic reactions taking place during Ab maturation in B cells, and performing these reactions successfully in a test tube to generate individual monoclonal Abs.

Our technology also allows a conversion of any available Ab library with low complexity to a highly diverse Ab library by human AID and pol η. High complexity of Ab libraries is the key for success in screening and isolation of Abs with a high specificity against any chosen antigen. There are potentially major clinical advantages of using AID and pol η versus widely used random mutagenesis. AID and pol η generate IgV mutations that mimic natural SHM mutations in activated B-cells. For this reason, Abs generated by AID and pol η are likely to be highly tolerated by the human immune system. In contrast, random mutagenesis will introduce many Ab variants that likely elicit human immune response.

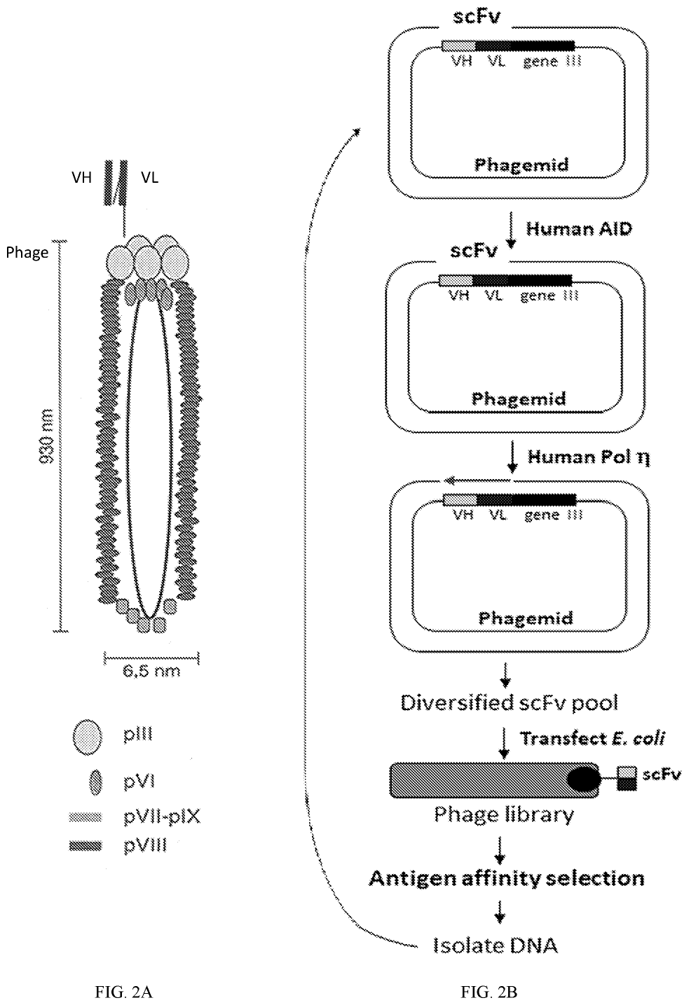

Described herein is a broad overview of our approach to manufacture monoclonal antibodies in a test tube. The Inventors have achieved strong proof-of-principle by making new unrelated monoclonal antibodies against different targets, i.e., monoclonal Abs that bind tightly to four Ags: 1) a membrane bound G-protein: a G-protein coupled receptor (GLP1R); 2) a human fatty acid amide hydrolase (FAAH) protein; 3) an artemin protein; and 4) activation-induced deoxycytidine deaminase (AID): a purified AID protein. The method for generating the four new antibodies above used AID enzyme, along with DNA polymerase η, as described in FIG. 2B.

In various embodiments, AID and polymerase η are the key mutagenic “reagents” to mutate antibody gene segments to generate monoclonal antibodies against virtually any antigen target. Examples are provided inducing hypermutation by AID and Pol η and using affinity maturation to screen and select for high-affinity Abs targeting G-coupled protein receptor (GLP1R), Fatty acid amide hydrolase (FAAH), artemin, and human AID.

In various embodiments of the methods, diversification or induction of hypermutation using AID and Pol η is performed in two or more stages, each stage repeatable for one or more rounds. A first stage can take place in the phage or phagemid clones with IgV genes, to generate a phage library or phagemid library; with which, initial phage or phagemid clones with (some) affinity to an antigen can be identified. A second stage can take place in the affinity maturation process based on the identified initial phage or phagemid clones, wherein further mutagenesis of the initial clones is induced using AID and Pol η to identify clones with high affinity to an antigen. In some instances, a third timing of mutagenesis can take place with naïve IgV genes, e.g., before introduction into a phage or phagemid.

Further embodiments provide that the methods include panning against an antigen after one or more stages, or in between rounds within a stage. In some aspects, panning is carried out against antigens expressed on cell surface. In some aspects, panning is carried out against purified antigens. In some aspects, earlier panning is carried out against cells whose surface express an antigen of interest, and later panning is carried out against purified or isolated form of the antigen. Independent in each stage, panning can be performed for one time, or repeated for a total of two times, three times, four times, five times, six times, seven times, or eight times, or more as needed by users.

Various embodiments provide methods involving the use of AID and the use of Pol η to induce mutagenesis or hypermutation in a test tube, excluding B-cells and T-cells. In some aspects, the incubation, contact, or reaction of a DNA substrate (e.g., IgV gene mix) with AID is in a cell-free environment. In some aspects, the incubation, contact, or reaction of a DNA substrate (e.g., IgV gene mix, or phage or phagemid clones displaying an IgV or linked IgVs, e.g., scFv) is in an environment without B cells, with no B cells and T cells, or without a mammalian cell.

Further embodiments provide that the AID used in the methods is recombinant AID, or isolated AID. In some embodiments, the AID is pre-treated with RNase to remove inhibitor RNA bound to AID. In some embodiments, the AID is a GST fusion protein (GST-AID), and can be made by amplifying AID cDNA from Ramos B cell mRNA, cloning it into a vector, and expressing the vector in cell cultures to produce recombinant AID.

In some embodiments, incubating or contacting a DNA substrate (e.g., IgV genes mix, or phage or phagemid clones displaying IgVs or IgV fusions) with AID to induce deamination (mutagenesis) includes mixing AID, RNase, and the DNA substrate. In some embodiments, incubating or contacting a DNA substrate with AID to induce deamination includes heat-denaturing the DNA substrate (e.g., above Tm to separate the strands), cooling the denatured DNA substrate (e.g., to 37° C. or an appropriate temperature range 25-40° C. for AID enzymatic activity), and immediately mixing the cooled DNA substrate with pre-activated AID. In further embodiments, deamination reaction is terminated by extracting the reaction mixture with a solution comprising phenol, chloroform and isoamyl alcohol for one time, two times, three times or more.

Further embodiments provide that the Pol η used in the methods is recombinant Pol η, isolated Pol η, or purified Pol η. In some aspects, the Pol η is human Pol η. In some aspects the Pol η is mouse Pol η. And in some aspects, the Pol η is mammalian Pol η. In various embodiments, gapped DNA substrates are incubated with Pol η to initiate DNA synthesis (in the presence of four dNTPs) with base substitution errors, wherein the gap in one strand of the DNA substrate exposes a segment of the other strand, preferably this segment on the other strand comprising IgV genes and acting as the template for Pol η-mediated, error-prone synthesis to fill the gap. In some embodiments, the gapped DNA substrates are AID-treated IgV genes that have been annealed to primers. In other embodiments, a gap in the DNA substrates can be an overhang of a first IgV over a second IgV, e.g., staggered stacking of annealed product between a first IgV and a second IgV due to complementary sequences introduced to opposing ends of the IgV (e.g., 3′ end of VH and 5′ end of Vk or Vl introduced with complementary sequences; or 5′ end of VH and 3′ end of Vk or Vl introduced with complementary sequences), so the overhanging portion of one IgV can act as template while the other as primer for Pol η extension. In further embodiments, error-prone synthesis with Pol η is terminated by extracting the reaction mixture with a solution comprising phenol, chloroform and isoamyl alcohol for one time, two times, three times or more. Subsequently, DNA products can be ligated with a phage or phagemid vector, which then can be introduced/transfected/electroporated into a microbe (e.g., Escherichia coli).

Described herein is an in vitro method, including providing a library of phage clones, diversifying the library of phage clones by contacting the library of phage clones with AID, further diversifying the library of phage clones by contacting the library of phage clones with low fidelity pol η, and transfecting the diversified library into a microbe, and generating a phage library.

In other embodiments, the method includes providing a library of phagemid clones, diversifying the library of phagemid clones by contacting the library of phagemid clones with activation-induced deoxycytidine deaminase (AID), further diversifying the library of phagemid clones by contacting the library of phagemid clones with DNA polymerase eta (Pol η), and transfecting the diversified library into a quantity of bacteria, and generating a phage library.

In various aspects, the phage or phagemid clones are vectors for expressing one or more IgV genes (e.g., scFv, Fab, VHH) on the surface of a filamentous phage, comprising a cassette for expressing the one or more IgV polypeptides/proteins. In further aspects, the one or more IgV genes in the cassette are AID and Pol f-mutagenized IgV genes. The cassette includes upstream and downstream translatable DNA sequences operatively linked via a sequence of nucleotides adapted for directional ligation of an insert DNA, i.e., a polylinker, where the upstream sequence encodes a prokaryotic secretion signal, and the downstream sequence encodes a filamentous phage protein.

In further embodiments, the method additionally includes panning the phage library against an antigen. In other embodiments, the method includes diversifying the library of phage clones, further diversifying the library of phage clones, transfecting the diversified library into a microbe, generating a phage library, and panning the phage library is repeated one or more times.

In other embodiments, the library of phage clones is made by a method, including providing a library of IgV genes, diversifying the library of IgV genes by contacting the library of IgV genes with AID, further diversifying the library of IgV genes by contacting the library of IgV genes with Pol η, generating a phage library, panning the phage library against at least one antigen to generate the library of phage clones. In some aspects, the library of IgV genes is a library of human naïve VH genes, VL genes, or both. In some aspects, the library of IgV genes is a library of human naïve IgV genes obtained from tonsil B cells. In some aspects, the library of IgV genes is a library of llama naïve VHH genes. In some aspects, the library of naïve IgV genes are human germline sequences, cover the human immune repertoire, and/or have high sequence identity (at least 99%, 98%, 97%, 96%, 95%, 90%, 85%, 80%, or 75%) to the natural derived human antibody V genes. In some aspects, the library of naïve IgV genes are rearranged V genes from B-cells of non-immunized donors, who did not have any previous contact with a specific antigen. In other aspects, the library of IgV genes is an immune library derived from immunized donors. In other aspects, the library of IgV genes is a synthetic library based on a human V gene framework with randomly integrated complementarity-determining region (CDR) cassettes. In other aspects, the library of IgV genes is a semisynthetic library derived from unrearranged V genes or from one antibody framework with genetically randomized CDR3 regions.

In further embodiments, the library of phage clones is made by a method, including providing a library of naïve IgV genes, diversifying the library of naïve IgV genes by contacting the library of naïve IgV genes with activation-induced deoxycytidine deaminase (AID), further diversifying the library of naïve IgV genes by contacting the library of naïve IgV genes with DNA polymerase eta (Pol η), generating a phage library, panning the phage library against at least one antigen to generate the library of phage clones.

In other embodiments, the method includes diversifying the library of phage or phagemid clones, further diversifying the library of phagemid clones, or both, comprising formation of a single stranded DNA cassette to generate gapped DNA. In other embodiments, the method includes digesting dsDNA with a restriction enzyme, denaturing the digested DNA and a DNA containing a segment of an IgV gene or linked IgV genes (e.g., scFv) in a mixture (or combining separately denatured DNAs into a mixture), and annealing the mixture to generate gapped DNA wherein the segment of the IgV genes or linked IgV genes is exposed as a ssDNA segment. In further aspects, if the DNA containing a segment of an IgV gene or linked IgV genes has been digested with another restriction enzyme, the annealed mixture would generate double gapped DNA.

Further embodiments provide a method for screening a phage or phagemid library for antibody fragments, comprising (a) providing a diversified phage or phagemid library, which comprises recombinant phage vectors or phagemid vectors, wherein IgV genes or antibody fragments treated with AID and Pol η before, after, or both before and after recombinantly constructed into the phage or phagemid vectors; (b) expressing the IgV genes or antibody fragments in said phage or phagemid library wherein said IgV genes or antibody fragments are expressed in bacterial host cells, and where said host cells comprise the recombinant phage vector or phagemid vector, and nucleic acid sequences encoding a coat protein (e.g., pIII, pVIII, or pIX) of filamentous phage fused to a scFv or VHH or Fab amino acid sequence, wherein said scFv or VHH or Fab amino acid sequence binds to an antigen; and (c) selecting bacterial cells expressing said scFv or VHH or Fab antibody fragments having an binding specificity or affinity (e.g., above a reference or control, or threshold) to an antigen (e.g., a selected antigen, or an antigen of interest, or a binding partner). In some embodiments, a method for screening a phage or phagemid library further comprises washing away unbound IgV genes or antibody fragments.

In some embodiments, a reference/control can be an empty assay device, e.g., an empty well; or a background level from the assay device. In some embodiments, a reference/control can be a level of binding to a non-specific protein, such as bovine serum albumin, in the assay. In some embodiments, a threshold is a level that is 3, 4, 5, 6, 7, 8, 9, 10, 15, or 20 times as high as, or even greater, compared to a background level or compared to a level of binding in the presence of a non-specific protein. In other embodiments, a threshold is the level of binding by a known antibody to the same antigen. In further embodiments, a threshold is 90%, 80%, 70%, 60%, 50%, 40%, 30%, 20%, or 10% of the level of binding by a known antibody to the same antigen. In further embodiments, a threshold is at least 100%, 110%, 120%, 130%, 140%, 150%, 160%, 170%, 180%, 190% or 200% of the level of binding by a known antibody to the same antigen.

In various aspects, phage or phagemid libraries are prepared in a stock with a high titer, at least 1×10¹¹, 5×10¹¹, 1×10¹², 5×10¹², 1×10¹³ or 5×10¹³ TU/ml for the panning.

In various aspects, successive rounds of panning identify antibody fragments (e.g., scFv, Fab, VHH) with a high affinity, with Kd values in the low nanomolar or in the picomolar range, e.g., 300-500 nM, 200-300 nM, 100-200 nM, 50-100 nM, 10-50 nM, 1-10 nM, or 0.1-1 nM. In some aspects, a further round of panning improves the affinity by at least 2-fold, 3-fold, 4-fold, 5-fold, or greater, compared to a previous round. In some aspects, with four rounds or less (e.g., 4, 3, 2 rounds or 1 round) of panning, selected (affinity-matured) antibody fragments generated by the methods disclosed herein has a 2-fold, 2.2-fold, 2.4-fold, 2.6-fold, 2.8-fold, 3-fold, 1.8-fold, 1.6-fold, 1.4-fold or 1.2-fold increase in affinity compared to parent antibody fragment (before the AID and Pol η affinity maturation) towards the same antigen.

An antigen of choice can be used in the panning to identify bound DNA substrate(s), phage(s), phagemid(s). In some embodiments, the antigen is GLP1R. In some embodiments, the antigen is FAAH. In some embodiments, the antigen is artemin. In some embodiments, the antigen is AID. In some embodiments, the antigen is a spike (S) protein, or a nucleocapsid (N) protein, of a coronavirus (e.g., SARS-CoV-2). In some embodiments, the antigen is a virus, a bacterium or a fungus. In some embodiments, the antigens suitable in the bio-panning in the disclosed methods include but are not limited to antigens expressed on B-cells, antigens expressed on carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases. In some embodiments, the antigens are specific for cancer, inflammatory disease, neuronal-disorders, diabetes, cardiovascular disease, infectious diseases or a combination thereof.

Antigens specific for cancer include but are not limited to any one or more of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2 or vimentin.

Antigens specific for inflammatory diseases include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, Lama glama, LFA-1 (CD11a), MEDI-528, myostatin, OX-40, rhuMAb β7, scleroscin, SOST, TGF beta 1, TNF-α or VEGF-A.

Antigens specific for neuronal disorders include beta amyloid or MABT5102A. Antigens specific for diabetes include but are not limited to any one or more of L-1β or CD3. Antigens specific for cardiovascular diseases include but are not limited to any one or more of C5, cardiac myosin, CD41 (integrin alpha-IIb), fibrin II, beta chain, ITGB2 (CD18) and sphingosine-1-phosphate. Antigens specific for infectious diseases include but are not limited to any one or more of anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-α.

Described herein is a phage library made by providing a library of phagemid clones, diversifying the library of phagemid clones by contacting the library of phagemid clones with AID, further diversifying the library of phagemid clones by contacting the library of phagemid clones with by contacting the library of phagemid clones with low fidelity DNA polymerase, and transfecting the diversified library into bacteria, and generating a phage library. In other embodiments, the method, includes providing a library of phage clones, diversifying the library of phage clones by contacting the library of phage clones with activation-induced deoxycytidine deaminase (AID), further diversifying the library of phage clones by contacting the library of phage clones with DNA polymerase eta (Pol η), and transfecting the diversified library into bacteria, and generating a phage library. In other embodiments, the method includes panning the phage library against an antigen. In other embodiments, the method includes diversifying the library of phage clones, further diversifying the library of phage clones, transfecting the diversified library into bacteria, generating a phage library, and panning the phage library repeated one or more times.

In other embodiments, the library of phage clones is made by a method, including providing a library of naïve IgV genes, diversifying the library of naïve IgV genes by contacting the library of naïve IgV genes with activation-induced deoxycytidine deaminase (AID), further diversifying the library of naïve IgV genes by contacting the library of naïve IgV genes with DNA polymerase eta (Pol η) so as to generate a library of diversified IgV genes, generating a phage library that can express diversified IgV genes, panning the phage library against at least one antigen to generate the library of phage clones. In various embodiments, generating a phage library (or constructing such expression libraries and nucleic acids encoding them) uses known cloning techniques described in the art, and any known expression library or a newly developed expression library can be used. For example, such libraries may consist of natural polypeptides or fragments thereof, or may be completely or partially random or synthetic. For example, libraries can be obtained by cloning nucleic acids of diversified IgV genes into a phage or phagemid vector. Further description of cloning techniques in generating phage libraries are described in WO2006038022 and WO2009085462, which are incorporated by reference.

Generally, the techniques used to obtain library designs will be based on genetic engineering methods. In this case, nucleic acid sequences encoding the actual protein or peptide, e.g., diversified IgV genes, which usually changes between the various elements of the library, thus providing a variety of libraries, are included in the expression vectors corresponding to the type of expression system used. Suitable expression vectors for use in phage display, covalent or non-covalent display, bacterial expression, and the like. If a phage display is selected as the expression library, either phage or phagemid vectors can be used. In some embodiments, phagemid vectors are used. In some embodiments, phage vectors are used.

A monoclonal antibody made by an in vitro method, including providing a library of phage clones, diversifying the library of phage clones by contacting the library of phage clones with AID, further diversifying the library of phage clones by contacting the library of phage clones with low fidelity Pol η, and transfecting the diversified library into bacteria, and generating a phage library. In other embodiments, the method includes providing a library of phagemid clones, diversifying the library of phagemid clones by contacting the library of phagemid clones with activation-induced deoxycytidine deaminase (AID), further diversifying the library of phagemid clones by contacting the library of phagemid clones with DNA polymerase eta (Pol η), and transfecting the diversified library into bacteria, and generating a phage library. In other embodiments, the method includes panning the phage library against an antigen. In other embodiments, the method includes diversifying the library of phagemid clones, further diversifying the library of phagemid clones, transfecting the diversified library into bacteria, generating a phage library, and panning the phage library repetitively one or more times. In other embodiments, the library of phage clones is made by a method, including providing a library of naïve IgV genes, diversifying the library of naïve IgV genes by contacting the library of naïve IgV genes with activation-induced deoxycytidine deaminase (AID), further diversifying the library of naïve IgV genes by contacting the library of naïve IgV genes with DNA polymerase eta (Pol η), generating a phage library, panning the phage library against at least one antigen to generate the library of phage clones. In other embodiments, the monoclonal antibody is a GLP1R monoclonal antibody. In other embodiments, the monoclonal antibody is a FAAH monoclonal antibody. In other embodiments, the monoclonal antibody is an artemin monoclonal antibody. In other embodiments, the monoclonal antibody is an AID monoclonal antibody.

A library of phage clones expressing diversified IgV genes is provided. A phage library is also provided by one or more of the methods described herein. Described herein is an in vitro method, including providing a library of phage clones, diversifying the library of phage clones by contacting the library of phage clones with AID, further diversifying the library of phagemid clones by contacting the library of phagemid clones with by contacting the library of phagemid clones with low fidelity Pol η, and transfecting the diversified library into bacteria, and generating a phage library. In other embodiments, the method includes providing a library of phagemid clones, diversifying the library of phagemid clones by contacting the library of phagemid clones with activation-induced deoxycytidine deaminase (AID), further diversifying the library of phagemid clones by contacting the library of phagemid clones with DNA polymerase eta (Pol η), and transfecting the diversified library into bacteria, and generating a phage library. In other embodiments, the method includes panning the phage library against an antigen. In other embodiments, the method includes diversifying the library of phagemid clones, further diversifying the library of phagemid clones, transfecting the diversified library into bacteria, generating a phage library, and panning the phage library repetitively one or more times. In other embodiments, the library of phage clones is made by a method, including providing a library of naïve IgV genes, diversifying the library of naïve IgV genes by contacting the library of naïve IgV genes with activation-induced deoxycytidine deaminase (AID), further diversifying the library of naïve IgV genes by contacting the library of naïve IgV genes with DNA polymerase eta (Pol η), generating a phage library, panning the phage library against at least one antigen to generate the library of phagemid clones.

An antibody, a single-chain variable fragment (scFv), a single domain antibody, a VHH antibody (nanobody), or a nanobody-based human heavy chain antibody, and/or an antigen-binding fragment thereof are provided, which are produced by one or more of the methods described herein.

In some embodiments, an anti-human glucagon-like peptide-1 receptor (GLP1R) antibody, scFv, single domain antibody (or nanobody), or nanobody-based human heavy chain antibody is provided, which comprises a variable heavy (VH) chain having a peptide sequence of SEQ ID NO:112 or encoded by a DNA sequence of SEQ ID NO:111, and, for the antibody and scFv, a variable light (VL) chain having a peptide sequence of SEQ ID NO: 114 or encoded by a DNA sequence of SEQ ID NO:113.

In some embodiments, an anti-human AID antibody, scFv, single domain antibody (or nanobody), or nanobody-based human heavy chain antibody is provided, which comprises a VH chain having a peptide sequence of SEQ ID NO:120 or encoded by a DNA sequence of SEQ ID NO:119, and, for the antibody and scFv, a VL chain having a peptide sequence of SEQ ID NO: 122 or encoded by a DNA sequence of SEQ ID NO:121.

In some embodiments, an anti-artemin variable domain of heavy chain is provided, which has a peptide sequence of SEQ ID NO:118 or encoded by a DNA sequence of SEQ ID NO:117. In some embodiments, llama's nanobody against artemin is provided, which has a variable domain of llama heavy chain antibody (VHH) having a peptide sequence of SEQ ID NO:118 or encoded by a DNA sequence of SEQ ID NO:117.

In some embodiments, an anti-fatty acid amide hydrolase (FAAH) variable domain of heavy chain is provided, which has a peptide sequence of SEQ ID NO:116 or encoded by a DNA sequence of SEQ ID NO:115. In some embodiments, llama's nanobody against FAAH is provided, which comprises a VHH having a peptide sequence of SEQ ID NO:116 or encoded by a DNA sequence of SEQ ID NO:115.

Described herein is a kit, including a quantity of naïve IgV genes, a quantity of activation-induced deoxycytidine deaminase (AID), a quantity of low fidelity low fidelity Pol η, and instructions for use. In various embodiments, instructions for use in the kit include one or more, or all, steps of: (1) producing gapped, double-stranded DNA substrates wherein the naïve IgV genes are exposed as single-stranded segment in the gapped, double-stranded DNA (e.g., by digesting dsDNA with or without the IgV genes, denaturing them in a mixture, and reannealing the mixture, to form gapped, dsDNA), (2) pretreating the AID to activate it, (3) combining/contacting/incubating the gapped DNA with an effective amount of AID for deamination, (4) further combining/contacting/incubating the gapped DNA with an effective amount of Pol η for synthesis to close or fill in the lesion or gap in the gapped DNA, and (5) terminating the deamination and/or the synthesis by extracting reaction mixture with a solution containing phenol, chloroform and isoamyl alcohol. In further embodiments, the instructions can include details as described in Examples section.

Described herein is a kit, including a library of phage or phagemid clones, a quantity of activation-induced deoxycytidine deaminase (AID), a quantity of low fidelity DNA polymerase eta (Pol η), and instructions for use. In various embodiments, instructions for use in the kit include one or more, or all, steps of: (1) producing or obtaining gapped, double stranded library of phage or phagemid clones, (2) pretreating the AID for activation, (3) combining/contacting/incubating the gapped, double stranded clones with an effective amount of AID for deamination, (4) further combining/contacting/incubating the gapped, double stranded clones with an effective amount of Pol η for synthesis to close or fill in the lesion or gap in the gapped DNA, and (5) terminating the deamination and/or the synthesis by extracting reaction mixture with a solution containing phenol, chloroform and isoamyl alcohol.

### EXAMPLES

### Example 1. Highly Diversified Mutation Spectra Catalyzed by AID and Pol η on IgV and on scFv Library In Vitro

### I. AID and Pol η-Induced Mutations in Human IgHV3-23*01 Gene:

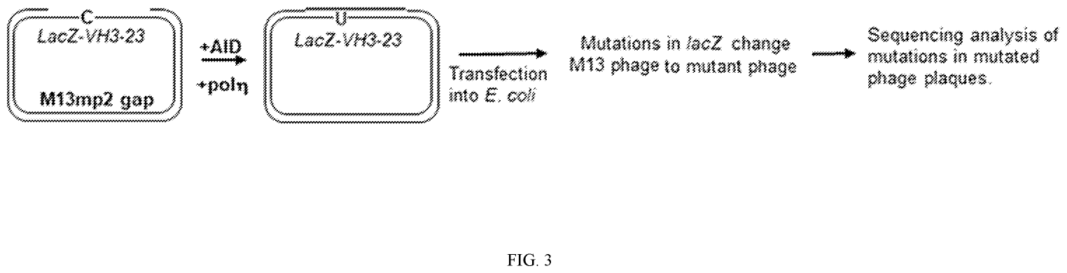

In B-cells, SHM is generated at G-C sites by AID and at A-T sites by Pol η. Our comparison of AID actions on human and murine IgV sequences in vitro and in vivo has provided the impetus and scientific rationale to implement Ab diversification strategies using AID and Pol η. (Note that in vitro here means a biochemical defined system. In vivo here means within cells or organisms). To explore the application of AID and Pol η in IgV affinity maturation in test tube, we have examined their combined mutagenic actions on a human IgHV3-23*01 gene, as an example of any protein sequence that can be varied. IGHV3-23*01 is the most commonly used variable region during normal immune responses and in chronic lymphocytic leukemia. We have constructed a M13mp2 phage derivative with IgHV3-23*01 inserted downstream of lacZ gene. Partial DNA gapped substrates with the lacZ-IgHV3-23*01 region as ssDNA (FIG. 3; as the circular DNA is gapped, a single-stranded region is created). The lacZ-IgHV3-23*01 region was first exposed to AID deamination action for 5 min at 37° C. followed by gap-filling synthesis by purified human Pol η for 2 h in the presence of 500 μM dNTP. The DNA products were transfected into E. coli cells and plated on μ-complementation host cells (CSH50 strain) in the presence of X-gal and IPTG. AID deamination within the lacZ-IgHV3-23*01 ssDNA gap region causes C to T mutations, whereas Pol η error prone gap-filling synthesis causes other mutations. Mutation(s) in lacZ-IgHV3-23*01 region gives rise to either white or light blue mutant M13 plaques. DNAs from mutant M13 phages were isolated and the entire IgHV3-23*01 insert region was sequenced. We have sequenced ˜150 mutant phage clones and compiled a total of 972 individual mutations in one mutation spectrum. The spectrum revealed that AID and Pol η add base substitution mutations throughout the IgV region, with C→T mutations are assumed to arise from AID deamination, while the rest of mutations (none C to T) were assumed to be added by Pol η. Mutations occur at WRC hotspot sites for AID and WA hotspot sites for Pol η. There are also mutations found at other non-canonical hotspot sites. We also observed clusters mutations (i. e. multiple mutations in a small region) in individual clones. The formation of mutation clusters at canonical WRC and WA somatic hypermutation hotspot motifs and the broad distribution of mutations across entire human IgHV3-23*01 gene indicate that both AID and Pol η retain their signature activities, and hence they can be used for in vitro V gene affinity maturation to mimic in vivo IgV mutagenic diversification in B-cells.

II. Optimized Procedures for In Vitro Affinity Maturation of Ab scFv Library:

The combined actions of AID and Pol η on IgHV3-23*01 (shown above) indicated that they are suitable for V gene library diversification in vitro (and any protein sequence that one wishes to vary for any purpose). As a proof of principal for the application of AID and Pol η actions to diversify an existing Ab scFv library (or sub-library), we have constructed a “mini” synthetic scFv library using 18 VH and 20 VL chains. Random combination of VH and VL chains (360 possible combinations) to form scFv (VH joins to VL via a linker (G₄S)₄ (SEQ ID NO:1) was achieved by overlapping PCR due to the presence of 15 complementary bases in the linker region between the 3′ end of VH and the 5′ end of VL. scFv pool was cloned into pADL-20c phagemid vector and scFv phage library was obtained by transformation of ligated DNA into E. coli TG1 cells.

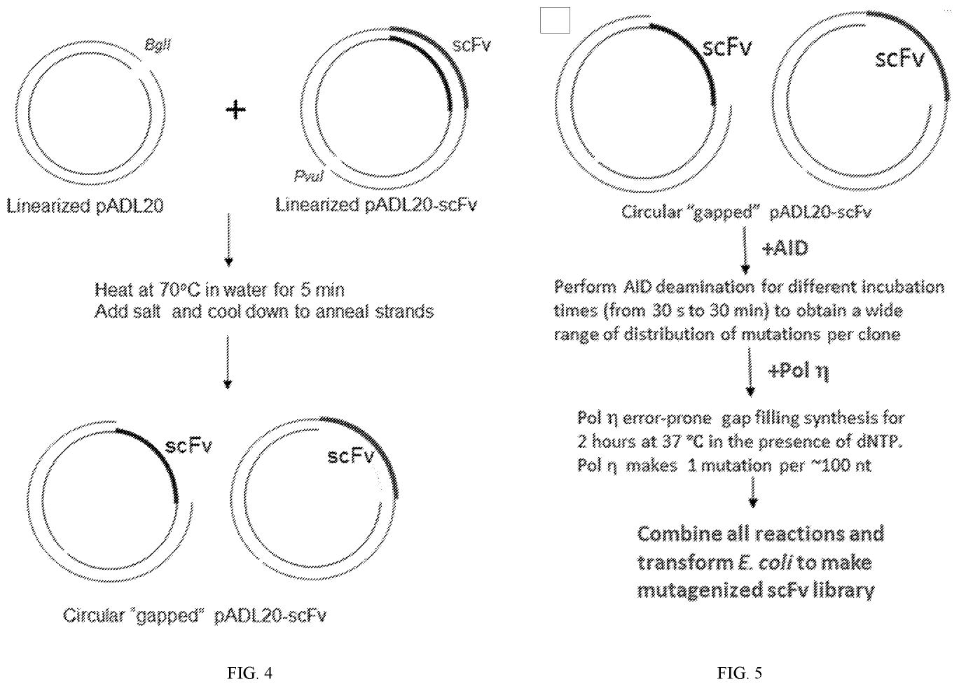

In order to subject both DNA strands of IgV to hypermutation by AID and Pol η, we have developed an improved “double gap” method to construct partially dsDNA construct with scFv gene fragment exposed as ssDNA region (FIG. 4). Briefly, a phagemid dsDNA without scFv (pADL20) is linearized with BglI restriction enzyme and the DNA containing a scFv insert (pADL20-scFv library) is linearized with PvuI. The linearized DNAs were denatured in water at high temperature (70° C.), quickly mixed together and cooled down in the presence of salt to formed circular gapped constructs with the scFv region exposed as ssDNA on the sense or anti-sense strand). The efficiency of gapped construct formation was examined by transformation of 10 pg of DNA into E. coli competent cells by electroporation. Linearized DNA (both pADL20 and pADL20-scFv) only gave rise to ˜9 to 53 colonies, whereas following denaturing and annealing, there are >30-fold increase in the number of colonies, indicating a very efficient circular gapped scFv formation.

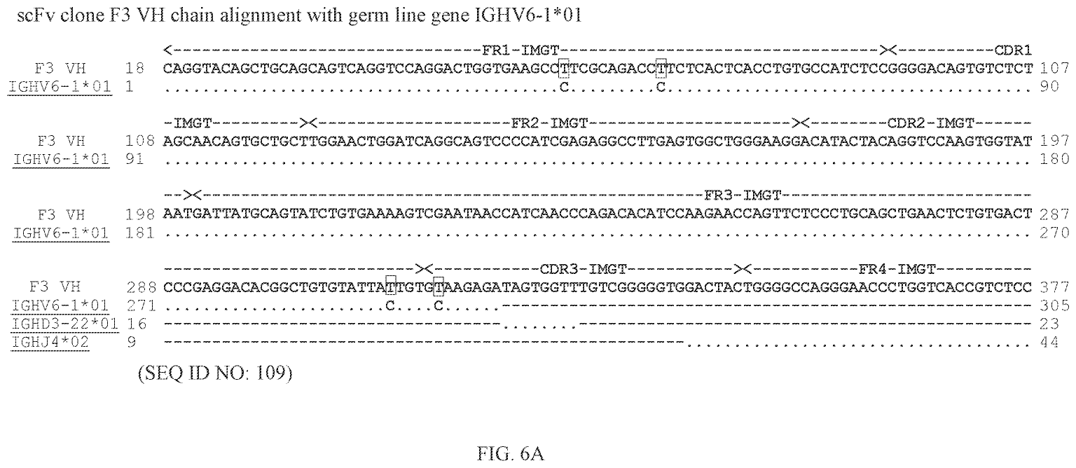

We have used the circular “gapped” pADL20-scFv library to perform the in vitro affinity maturation procedure (FIG. 5). Previous biochemical studies have shown that AID is a very inefficient ssDNA scanning enzyme with the deamination rate of ˜1 deamination per 40 s and that the average number of deaminations on individual substrates proportionally increases with increasing incubation times. However, there is a wide variation in the distribution regarding the numbers of mutations per clone at any incubation time, ranging from 2 to >70 mutations/clone for the lacZ target. To maximize the scFv diversity, the gapped constructs were incubated with AID for different incubation times (from 1 to 30 min). The deaminated gapped DNAs were then subjected to Pol η error-prone gap filling synthesis for 2 h. Pol η is highly error-prone and on average makes 1 mutation per 100 nucleotide incorporation while favoring WA hotspot motifs. Following AID and Pol η treatment, all DNAs were pooled and used to transform E. coli cells (MC1061 ung− strain) to make an AID and Pol η-mutagenized scFv gene library (FIG. 5). We have randomly picked individual transformants and sequenced heavy (V_{H}) and light (V_{L}) chains of scFv. Table 1 of results for sequenced clones showed a broad distribution in number of mutations per clone ranging from 0 to 33 mutations for the V_{H} and 0 to 22 mutations for V_{L}. The total number of mutations for whole scFv (VH+VL) ranges from 0 to 52 mutations per scFv clone. In addition, there are clones with multiple C to T mutations and there are clones with multiple G to A mutations, as well as mutations that are not C to T, or G to A, indicating that both AID and Pol η have access to both the sense and anti-sense strands and make a wide range of diversified mutations on scFv gene fragments.

TABLE 1
The distribution of mutations in 24 randomly
sequenced representative scFv clones.

| | | | | | Total number |
|---|---|---|---|---|---|
| Random | Heavy chain | | Light chain | | of VH + VL |
| clone | VH | Mutations* | VL | Mutations | mutations |
| 1 | VH933 | 1 | VL1082 | 1 | 2 |
| 2 | VH932 | 2 | V11059 | 0 | 2 |
| 3 | VH1086 | 2 | VL984 | 0 | 2 |
| 4 | VH1059 | 0 | VL1068 | 18 | 18 |
| 5 | VH1084 | 11 | VL984 | 0 | 11 |
| 6 | VH985 | 0 | VL914 | 0 | 0 |
| 7 | VH1082 | 3 | V1930 | 0 | 3 |
| 8 | VH1084 | 24 | V11083 | 0 | 24 |
| 9 | VH1085 | 0 | V1914 | 18 | 18 |
| 10 | VH1080 | 21 | VL985 | 1 | 22 |
| 11 | VH933 | 10 | V1914 | 1 | 11 |
| 12 | VH985 | 1 | VL984 | 1 | 2 |
| 13 | VH984 | 17 | VL933 | 1 | 18 |
| 14 | VH1084 | 11 | V1985 | 2 | 13 |
| 15 | VH1086 | 8 | V11083 | 4 | 12 |
| 16 | VH1081 | 0 | VL914 | 10 | 10 |
| 17 | VH1059 | 3 | V1931 | 1 | 4 |
| 18 | VH985 | 1 | VL1080 | 2 | 3 |
| 19 | VH1080 | 17 | V1930 | 22 | 39 |
| 20 | VH1082 | 17 | V11066 | 0 | 17 |
| 21 | VH932 | 33 | VL1083 | 19 | 52 |
| 22 | VH933 | 1 | V11084 | 17 | 18 |
| 23 | VH1082 | 4 | V1983 | 5 | 9 |
| 24 | VH1059 | 2 | VL1066 | 0 | 2 |

*Number of mutations compared to the aligned V gene.

Materials. M13mp2 phage and E. coli CSH50 and MC1061 ung− strains are from our lab collection. pADL20c phagemid vector, E. coli TG1 strain were purchased from Antibody Design Labs (San Diego, CA). A derivative of M13 mp2 phage with two unique restriction endonuclease sites for Pst1 and BglII downstream of lacZα gene (positions +281 and +293, respectively) was constructed by site directed mutagenesis. Human IgHV3-23*01 sequence was cloned into the Pst1 site in the forward orientation downstream of the lacZα gene. M13 phage were propagated in CSH50 host cells in 2×YT medium at 37° C. M13 phage dsDNA and ssDNA were purified using Maxiprep plasmid and QIAprep M13 ssDNA kits (Qiagen). DNAs were stored in 10 mM Tris (pH 8.5) at −20° C. Minimal medium plates for M13 phage were prepared by addition of 20 ml of 50×VB salt (MgSO₄·7H₂O—10 g, Citric Acid—100 g, K₂HPO₄—500 g, Na₂(NH₄)HPO₄·4H₂O—175 g dissolved in 1 L of H₂O), 20 ml of 20% glucose, 1 ml of Thiamine (5 mg/ml) and 2 ml of of Isopropyl β-D-1-thiogalactopyranoside (IPTG, 100 mM) to 1 L of autoclaved agar (15 g of Bacto-agar in 1 L of H₂O).
AID Purification. Human AID was expressed as a glutathione S-transferase (GST) fusion protein in Sf9 insect cells using a baculovirus expression system. Sf9 insect cells were infected with the recombinant baculovirus expressing GST-AID with a MOI of 3 and harvested after 72 h. Collected cells were resuspended in the insect cell lysis buffer (10 mM Tris pH 7.5, 130 mM NaCl, 1% Triton X-100, 10 mM Sodium fluoride, 10 mM sodium phosphate, 10 mM Sodium pyrophosphate, 1 mM EDTA, 1 mM DTT and protease inhibitor) and lysed on ice for 30 min. Crude extract, containing soluble GST-AID was collected after centrifugation of the lysed cells at 15,000 rpm for 1 h. Crude extract was incubated with Glutathione Sepharose resin (GE Healthcare) at 4° C. for 4 h. After extensive washing with PBS buffer, GST-AID (˜50 kDa) was eluted from the resin using an elution buffer 10 mM Tris (pH 9.8), 500 mM NaCl, 1 mM EDTA, 1 mM DTT and 10 mM reduced glutathione. Eluted samples were dialyzed overnight in 20 mM Tris pH 7.5, 50 mM NaCl, 0.1 mM dithiothreitol, 1 mM EDTA, 10% glycerol and stored at −70° C.
Human Pol η purification. N-terminal His-tagged full-length human Pol η was expressed in E. coli. Collected cells from 5 L cultures (˜25 grams) were resuspended in 180 ml of lysis buffer (50 mM Tris pH 7.5; 500 mM NaCl, 20 mM Imidazole, 10% glycerol, 10 mM 2-mercaptoethanol and 4 tablets of complete protease inhibitor (Roche) and lysed by French press. After centrifugation at 20,000×g for 45 min, the supernatant was incubated with 7.5 ml Ni-NTA resin (Qiagen) for 30 min at 4° C. on a rotating platform for 30 min. The NTA resin was washed with 50 ml of wash buffer 1 (50 mM Tris pH 7.5; 1 M NaCl, 20 mM Imidazole, 10% glycerol, 10 mM 2-mercaptoethanol) followed by 50 ml of wash buffer 2 (10 mM Na-Phosphate pH 7.7, 500 mM NaCl, 20 mM Imidazole, 10% glycerol, 10 mM 2-mercaptoethanol). His-tagged Pol η was eluted with elution buffer (10 mM Sodium Phosphate pH 7.7, 500 mM NaCl, 200 mM Imidazole, 10% glycerol, 10 mM 2-mercaptoethanol). Pol η fractions were pooled and applied to a Superdex G200 26/60 gel-filtration column (GE Healthcare) using running buffer (20 mM Tris pH 7.5; 500 mM NaCl, 1 mM EDTA, 1 mM DTT, 5% glycerol). Peak fractions containing monomeric His-tagged Pol η were collected and diluted with 4 volumes of a dilution buffer (20 mM Sodium Phosphate pH 7.3, 10% glycerol, 1 mM DTT). The diluted pool was loaded into a 1 ml mono S ion-exchange column equilibrated with Buffer A (20 mM Na-Phosphate pH7.3; 100 mM NaCl, 10% glycerol, 10 mM 2-mercaptoethanol). After washing the column with 20 ml buffer A, his-Pol η was eluted using 20 ml gradient of 100 ml NaCl to 1000 mM NaCl in buffer A. Pure his-Pol η fractions were pooled, dialyzed overnight in a dialysis buffer (20 mM Tris, pH 7.5; 50 mM NaCl, 1 mM EDTA, 1 mM DTT, 10% glycerol) and stored at −70° C.
Construction of M13 gap construct with the lacZα-IgHV3-23*01 ssDNA region. Close circular DNA gapped substrates with the lacZα-IgHV3-23*01 region as ssDNA were constructed as follow. M13mp2 dsDNA was digested with PvuII and BglII restriction enzymes (New England Biolabs), separated by 0.7% agarose gel electrophoresis and ˜6.8 kb PvuII-BglII fragment was extracted and purified by Qiaquick Gel Extraction Kit (Qiagen). 500 ng of the PvuII-BglII fragment of M13mp2 was denatured in a PCR tube with 45 μl of H₂O at 70° C. for 5 min, followed by a quick addition of 250 ng of purified ssDNA of M13mp2 lacZα-IgHV3-23*01 and 5 μl of 20×SSC buffer (3 M NaCl, 300 mM Sodium citrate, pH 7.0). The mixture was incubated at 60° C. for 5 min and placed on ice. Gapped DNA from 8 to 16 PCR tubes were pooled, desalted 3 times with H₂O using Amicon Ultra-0.5 10 kDa (Millipore) centrifugal filter unit and stored in 1 mM Tris (pH 8.0) and 0.1 mM EDTA at −20° C.
AID deamination and Pol η error-prone gap-filling synthesis in vitro. AID deamination reactions (30 μl total volume), containing GST-AID (100 ng), RNase (100 ng) and a gapped DNA substrate (500 ng) dissolved in a reaction buffer (10 mM Tris-HCl, pH 8.0, 1 mM EDTA, 1 mM dithiothreitol), were carried out at 37° C. for 5 min and terminated by twice extracting the DNA product with phenol:chlorophorm:isoamyl alcohol (25:24:1). The deaminated gap DNA (1 μg) were subjected to Pol η gap-filling synthesis at 37° C. for 2 h in the presence of 40 mM Tris-HCl (pH 8.0), 50 mM NaCl, 2.5% glycerol, 10 mM Dithiothreitol, 2.5 mM MgCl₂, 500 μM each of the four dNTPs and 300 ng of human Pol f. Synthesis reaction was terminated and Pol η was removed by twice extracting the DNA product with phenol:chlorophorm:isoamyl alcohol (25:24:1). AID and Pol η treated DNA were desalted 4 times with H₂O using Amicon Ultra-0.5 10 kDa (Millipore) centrifugal filter unit.
Analysis of AID and Pol η-induced mutations in IgHV3-23*01 sequence in vitro. 50 ng of desalted DNA were incubated with 50 μl of uracil glycosylase deficient (ung⁻) MC1061 competent cells and transformation was carried out by electroporation using a BioRad electroporator. Following addition of 1 ml of SOC medium and incubation at 37° C. for 30 min, aliquots of electroporated cells (5 to 200 μl) were added to a tube containing 3 ml of soft agar (7.5 g of bacto-agar in 1 L of H2O; autoclave and keep at 42° C.), 250 μl of mid-log CSH50 α-complementation host cells, 50 μl of 5-bromo-4-chloro-3-indolyl-beta-D-galactopyranoside (X-gal, 50 mg/ml), 50 μl of Isopropyl β-D-1-thiogalactopyranoside (IPTG, 100 mM), mixed and poured on the top of a minimal medium plate. After plate incubation overnight at 37° C., wild-type (colorless) and mutant (light or dark blue) M13 phage plaques were counted. DNAs from mutant M13 phages were isolated and the entire IgHV3-23*01 region was sequenced by standard Sanger sequencing.
Construction of a mini synthetic scFv library. Synthetic human genes, VH (18 genes) and VL (20 genes) were synthesized by Integrated DNA Technologies IDT (Coralville, Iowa). Each VH gene contains a sequence at the 3′ end:

(SEQ ID NO: 2)
GGTGGTGGTGGTTCTGGTGGTGGTGGTTCTGGCGGCGGCGGCTCC

which encodes a left portion (three pentamers') of the (G₄S)₄ (SEQ ID NO:1) linker, and each VL gene contains a sequence at the 5′ end: GGCGGCGGCGGCTCCGGTGGTGGTGGATCC (SEQ ID NO:3), which encodes a right portion (two pentamers') of the (G₄S)₄ linker. V gene fragments also contain a BglI restriction site for cloning into pADL20c phagemid vector. Random fusion of VH to VL via a flexible (G4S)4 linker was carried out using overlapping PCR. PCR reaction (50 μl volume) was assembled with 5 ng of VH+VL gene pool, 100 ng each of VH-F (TACTCGCGGCCCAGCCGGCCA (SEQ ID NO:4)) and VL-R (TGG TGT TGG CCT CCC GGG CCA (SEQ ID NO:5)) primer, and 25 μl of 2×PCR master mix (Promega). After 25 cycles of PCR (94° C.—1 min; 94° C.—30 s, 55° C.—30 s, 72° C.—1 min for 25 cycles; 72° C. for 2 min), scFv PCR products were purified, digested with BglI (New England Biolabs) and ligated with dephosphorylated BglI-digested pADL-20c vector using T4 DNA ligase. Ligated DNA were transformed into E. coli TG1 cells and plated on LB plates with 0.2% glucose and ampicillin (100 μg/ml). After incubation overnight at 37° C., TG1 transformant colonies were harvested, resuspended in LB medium in the presence of 15% glycerol and stored at −80° C.
Phagemid scFv “double gap” construction. To make the scFv double-gap constructions, dsDNA phagemid pADL-20c and pADL20-scFv library were first digested with BglI and PvuI restriction enzymes, respectively. Linearized dsDNAs were cleaned and purified by QIAquick PCR purification kit (Qiagen). Linear pADL20c and pADL20-scFv (0.5 μg each in 45 μl of H₂O) were heat denatured at 70° C. for 5 min in separate PCR tubes and combined. After addition of 10 μl of 20×SSC buffer (3 M NaCl, 300 mM Sodium citrate, pH 7.0), the mixture was incubated at 60° C. for 5 min and placed on ice. Double gapped DNA from 8 tubes were pooled, desalted 3 times with H₂O using Amicon Ultra-0.5 10 kDa (Millipore) centrifugal filter unit and stored in 1 mM Tris (pH 8.0) and 0.1 mM EDTA at −20° C. To verify the efficiency of gap formation, 10 pg of each linear pADL20c, pADL20-scFv, and the double gap constructs were used to transform 50 μl of TG1 electrocompetent cells by electroporation. The transformation mixtures were plated on LB plates in the presence of 100 μg/ml of ampicillin and TG1 bacterial colonies were counted after incubation at 37° C. overnight.
AID and Pol η mutagenesis for scFv double-gap constructs. To maximize the scFv diversity, the gapped constructs were incubated with AID in a series of individual tubes for different incubation times (30 s, 45 s, 1 min, 2 min, 5 min, 10 min, 20 min and 30 min). Each AID deamination tube (30 μl total volume), contains 500 ng of scFv double gap constructs, GST-AID (100 ng), RNase (100 ng) in a reaction buffer (10 mM Tris-HCl, pH 8.0, 1 mM EDTA, 1 mM dithiothreitol) at 37° C. At the indicated time point for each tube (30 s, 45 s, 1 min, 2 min, 5 min, 10 min, 20 min or 30 min), AID deamination was stopped by twice extracting the reaction mixture with phenol:chlorophorm:isoamyl alcohol (25:24:1). Deaminated scFv DNAs were combined and purified by QIAquick PCR purification kit (Qiagen). The deaminated scFv double-gap DNA (1 μg) were subjected to in Pol η gap-filling synthesis at 37° C. for 2 h in the presence of 40 mM Tris-HCl (pH 8.0), 50 mM NaCl, 2.5% glycerol, 10 mM Dithiothreitol, 2.5 mM MgCl₂, 1 mM dNTPs and 300 ng of human Pol η. Synthesis reaction was terminated and Pol η was removed by twice extracting the DNA product with phenol:chlorophorm:isoamyl alcohol (25:24:1). AID and Pol η treated DNA were desalted 4 times with H₂O using Amicon Ultra-0.5 10 kDa (Millipore) centrifugal filter unit and stored in 1 mM Tris (pH 8.0), 0.1 mM EDTA at −20° C. To analyze AID and Pol η actions on the scFv sequence, the purified double gap constructs were used to transform 50 μl of TG1 electrocompetent cells by electroporation. The transformation mixtures were plated on LB plates in the presence of 100 μg/ml of ampicillin and incubated at 37° C. overnight. pADL20-scFv phagemid from individual transformants were isolated and scFv regions were sequenced by Sanger sequencing. Sequence alignment and mutation analysis were performed using Sequencer program (Gene Code Corp.).

### Example 2. Construction of AID and Pol η Diversified Human ScFv and Llama VHH Phage Display Libraries

Ab phage display has become a robust technology for generation of Abs. The key for a successful isolation of antibodies for specific antigens is the antibody gene library used for the selection. The ability to diversify variable regions (IgV) in the test tube provides a natural way (i. e. similar to IgV diversification in B-cells) to increase the size and complexity of existing or “to-be-generated” human Ab repertoire libraries. Such Ab libraries would provide a powerful general “toolkit” for important research, diagnostic and therapeutic purposes.

Many types of Ab libraries have been constructed: immune, naïve, synthetic and semisynthetic. Immune libraries are derived from immunized donors. Due to obvious ethical reasons, there are limited numbers of available human immune libraries. Immune libraries are usually used in animals to obtain Abs against interested target antigens. Naïve Ab libraries constructed from rearranged V genes from B-cells of non-immunized donors, who did not have any previous contact with a specific antigen. Synthetic Ab libraries are based on a human V gene framework with randomly integrated complementarity-determining region (CDR) cassettes. Semisynthetic Abs libraries are derived from unrearranged V genes or from one antibody framework with genetically randomized CDR3 regions.

We have incorporated AID and Pol η-mediated V gene diversification steps during construction of human Ab gene libraries to make a relatively large scFv phage display library (˜1×10⁹ independent scFv phage clones). Our method of in vitro affinity maturation offers the following advantages over random mutagenesis and synthetic Ab libraries:
1. Ability to quickly increase the size and complexity of any naïve or immune Ab libraries;
2. IgV gene sequences have evolved to optimize efficient mutagenesis by AID and Pol η. Application of AID and Pol η to diversify V genes allows the production of minimal numbers of non-productive V gene variants;
3. There are potentially major clinical advantages of using AID and Pol η versus random mutagenesis. AID and Pol η-generate IgV mutations that mimic natural SHM mutations in activated B-cells. For this reason, Abs generated by AID and Pol η are likely to be highly tolerated by the human immune system. In contrast, synthetic libraries and random mutagenesis introduce many Abs variants that might elicit human immune response.
   I. Construction of a Mutagenized Human scFv Phage Display Library

A human scFv library in f3TR1 phage display vector (Type 3 trypsin release phage display vector f3TR1, GeneBank accession HM355479) was constructed in 4 steps: i, Heavy chain VH and light chain Vk and Vl gene repertoires were PCR amplified from tonsil B-cells cDNA using a set of subfamily-specific forward and reverse primers. In the second PCR, adaptor sequences were incorporated into the ends of VH and VL, which allow random fusion of VH and VL via a flexible (G₄S)₄ (SEQ ID NO:1) linker by overlapping PCR in a later step. BglI restriction sites are also added at the 5′ end of VH and 3′-end of VL for subsequent cloning into the phage vector; ii, V gene repertoires were denatured by heating at 95° C. for 2 min, quickly cooled down and incubated with purified AID at 37° C. to allow dC deaminations on both denatured ssDNA strands resulting in C to T or G to A mutations. Since a longer incubation time lead to a higher average number of AID-induced mutations on ssDNA substrates, various AID incubation times (30 s, 45 s, 1 min, 2 min, 5 min, 10 min and 20 min) were done in separate tubes and subsequently combined to increase the ranges of mutations on individual scFv; iii, AID-treated V genes were annealed to primers and one round of Pol η extension synthesis was carried out to introduce mutations at A and T sites; iv, Overlapping PCR was used to generate scFv gene repertoire. scFv PCR products were digested with BglI and directionally ligated into BglI sites of f3TR1 phage vector. Ligated DNA were transformed into MC1061 E. coli cells by electroporation. A total of 240 independent electroporations were carried out to generate a human f3TR1-scFv library composed of 1.1×10⁹ independent phage clones.

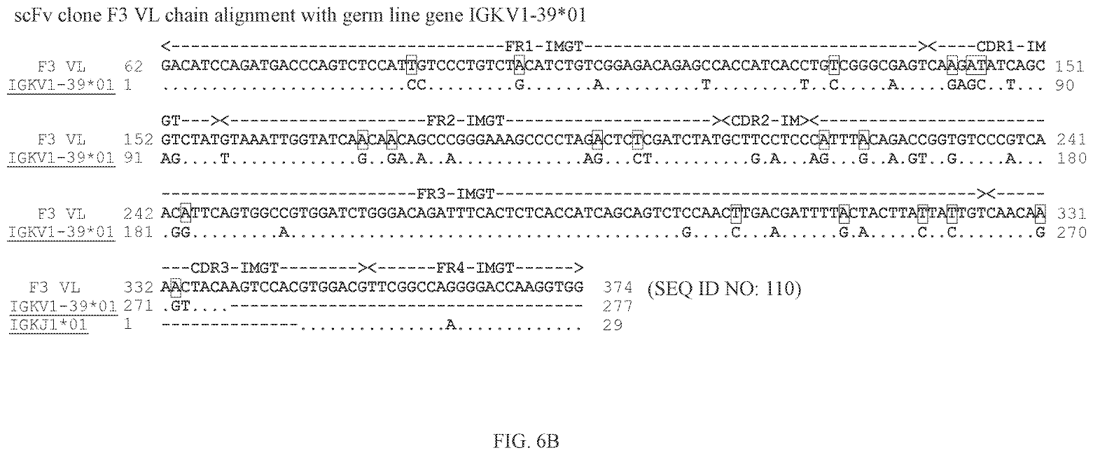

The diversity of the scFv repertoire and the quality of the primary library were examined by PCR analysis and by sequencing DNA segments encoding the scFv genes from 16 randomly picked f3TR1-scFv clones. Analysis of 16 clones by PCR using primers flanking the BglI sites showed that 15 clones (94%) contain an insert corresponding to the expected scFv size. Sequencing analysis of 16 scFv clones showed that all clones have different combination of heavy VH and light VL chains (Table 1). The variable regions were derived from 12 different V gene families, including five VH gene families (VH1, VH2, VH3, VH4, and VH6) and seven VL gene families of both kappa and lambda light chain (Vκ1, Vκ2, Vκ3, Vκ5 and Vλ1, Vλ3, Vλ6). The CDR3 of VH sequences were highly diverse, with lengths between 9 to 19 amino acids. The VL sequences had between 9 and 12 amino acids in their CDR3 regions (Table 2). Thus, the scFv gene fragments were distributed across the full repertoire of antibody germ line genes. The number of different bases from germ line genes varied from 0-32 mutations for VH and 0-48 mutations for VL. Only two V regions have no mutations indicating these V genes have not undergone affinity maturation in B-cells or in vitro (Table 2). Most of VH and VL in the library contain multiple mutations, indicating that these clones arose from antibodies produced in B-cells during secondary immune responses and/or during AID and Pol η affinity maturation in vitro. A representative scFv clone (F3) displaying a clear signature of AID-induced C to T and G to A mutations in vitro is shown in FIGS. 6A and 6B. Compared to the germ line genes, the heavy chain of this clone has 4 mutations, all C to T. The light chain contains 19 AID-induced C to T or G to A mutations along with other mutations, some of which can be attributed to Pol η (FIG. 6B). Actions of AID and Pol η on scFv in vitro are also seen by the presence of mutations in the linker (G₄S)₄ (SEQ ID NO:1) sequences. Since this linker sequence was added to the 2^{nd} PCR primer set to fuse VH and VL chains in the scFv library, mutations in the sequence are likely to arise from AID and Pol η treatment during the mutagenesis steps. Among 16 sequenced clones, 9 do not have any mutations in the linker sequence, 5 clones have an AID-induced C to T or G to A mutation and two clones contain Pol η-induced mutations that are not C to T or G to A.

The DNA sequence of VH in clone F3 (FIG. 6A):
(SEQ ID NO: 109)
CAGGTACAGCTGCAGCAGTCAGGTCCAGGACTGGTGAAGCCTTCGCAGAC
CTTCTCACTCACCTGTGCCATCTCCGGGGACAGTGTCTCTAGCAACAGTG
CTGCTTGGAACTGGATCAGGCAGTCCCCATCGAGAGGCCTTGAGTGGCTG
GGAAGGACATACTACAGGTCCAAGTGGTATAATGATTATGCAGTATCTGT
GAAAAGTCGAATAACCATCAACCCAGACACATCCAAGAACCAGTTCTCCC
TGCAGCTGAACTCTGTGACTCCCGAGGACACGGCTGTGTATTATTGTGTA
AGAGATAGTGGTTTGTCGGGGGTGGACTACTGGGGCCAGGGAACCCTGGT
CACCGTCTCC.
The DNA sequence of VL in clone F3 (FIG. 6B):
(SEQ ID NO: 110)
GACATCCAGATGACCCAGTCTCCATTGTCCCTGTCTACATCTGTCGGAGA
CAGAGCCACCATCACCTGTCGGGCGAGTCAAGATATCAGCGTCTATGTAA
ATTGGTATCAACAACAGCCCGGGAAAGCCCCTAGACTCTCGATCTATGCT
TCCTCCCATTTACAGACCGGTGTCCCGTCAACATTCAGTGGCCGTGGATC
TGGGACAGATTTCACTCTCACCATCAGCAGTCTCCAACTTGACGATTTTA
CTACTTATTATTGTCAACAAAACTACAAGTCCACGTGGACGTTCGGCCAG
GGGACCAAGGTGG.

TABLE 2
Sequencing analysis of representative clones from f3TR1-scFv phage display library.

| | Heavy chain | | | Light chain | | |
|---|---|---|---|---|---|---|
| Clone | VH | VH CDR3 | Mutations* | VL | VL CDR3 | Mutations* |
| F1 | IGHV3-23*04 | VRHRGKLRDGDNFE | 19 | IGLV1-50*01 | AGWDDSLSAWV (SEQ ID NO: 15) | 33 |
| | | APGDF | | | | |
| | | (SEQ ID NO: 14) | | | | |
| F2 | IGHV2-26*01 | AGRYSGSSGLDY | 20 | IGLV1-50*01 | ASWDDSLDAAI (SEQ ID NO: 17) | 23 |
| | | (SEQ ID NO: 16) | | | | |
| F3 | IGHV6-1*01 | VRDSGLSGVDY | 4 | IGKV1-39*01 | QQNYKSTWT (SEQ ID NO: 19) | 48 |
| | | (SEQ ID NO: 18) | | | | |
| F4 | IGHV6-1*01 | ARDPSGCYYGGTFDY | 0 | IGKV5-2*01 | LQRDTFPYT (SEQ ID NO: 21) | 15 |
| | | (SEQ ID NO: 20) | | | | |
| F5 | IGHV2-70*04 | ARSRSSGLYHFDL | 22 | IGLV6-57*01 | HSFDSGSQV (SEQ ID NO: 23) | 20 |
| | | (SEQ ID NO: 22) | | | | |
| F6 | IGHV2-26*01 | TDPNWTELVRP | 1 | IGLV3-21*01 | QVWDSGSNHMV (SEQ ID NO: 25) | 26 |
| | | (SEQ ID NO: 24) | | | | |
| F7 | IGHV3-21*01 | ARPGYSYGYDY | 5 | IGLV6-57*01 | QSSDSTNWV (SEQ ID NO: 27) | 13 |
| | | (SEQ ID NO: 26) | | | | |
| F8 | IGHV6-1*02 | ARTNGFDFWSGPPSV | 10 | IGLV3-19*01 | SSRDTRGKHLV (SEQ ID NO: 29) | 29 |
| | | DV | | | | |
| | | (SEQ ID NO: 28) | | | | |
| F9 | IGHV1-69*13 | ARYLDSSANFLGY | 5 | IGKV2-28*01 | MQNLQTPFT (SEQ ID NO: 31) | 7 |
| | | (SEQ ID NO: 30) | | | | |
| F10 | IGHV1-18*01 | AIGRDGYNLDFDY | 12 | IGKV1-39*01 | QQSYSTPWT (SEQ ID NO: 33) | 26 |
| | | (SEQ ID NO: 32) | | | | |
| F11 | IGHV3-33*01 | ARGTTHFDY | 13 | IGLV6-57*01 | QSYDSSNPWV (SEQ ID NO: 35) | 6 |
| | | (SEQ ID NO: 34) | | | | |
| F12 | IGHV2-5*02 | AHRLGMPRGTPYFDY | 27 | IGKV3-20*01 | QQYGHFPRT (SEQ ID NO: 37) | 7 |
| | | (SEQ ID NO: 36) | | | | |
| F13 | IGHV1-8*01 | VRFRPQTTTGDY | 32 | IGLV1-44*01 | ATWDDSVNGPG (SEQ ID NO: 39) | 26 |
| | | (SEQ ID NO: 38) | | | | |
| F14 | IGHV1-2*02 | ARQSGTQTEVTFDI | 21 | IGKV1-27*01 | QKYDGAPRT (SEQ ID NO: 41) | 4 |
| | | (SEQ ID NO: 40) | | | | |
| F15 | IGHV4-38- | AATYNWNDVGDY | 1 | IGKV5-2*01 | LQHDNFPLGP (SEQ ID NO: 43) | 0 |
| | 2*01 | (SEQ ID NO: 42) | | | | |
| F16 | IGHV6-1*01 | AKGIRGSTRSHDY | 5 | IGLV3-19*01 | NSRDSSGNHVV (SEQ ID NO: 45) | 12 |
| | | (SEQ ID NO: 44) | | | | |

*Numbers of mutations in scFv clones compared to germ line V genes

### II. Construction of Mutagenized Llama VHH-Phage Display Library

In addition to conventional Abs, llamas produce functional Abs that lack light chains and only contain the heavy chain. The variable domain of such a heavy chain Ab (VHH) contains 3 CDR regions and fully capable of antigen recognition. VHH is small (˜15 kD) has high sequence similarity to human VH3. The smaller size of VHH enables llama Abs to fit into epitopes that are normally not accessible to traditional antibodies. Studies have found VHH Abs to be very stable and highly soluble, which, together with their small size, makes them an ideal candidate for the generation of Ab libraries.

A llama mutagenized f3TR1-VHH phage library was constructed using an available naïve Llama VHH phage library (Abcore, Ramona, CA). Llama VHH repertoire was amplified from the naïve Llama VHH library using forward and reverse primers, which contain BglI restriction sites to allow subcloning VHH genes into f3TR1 phage vector. VHH gene library was sequentially mutagenized by AID and Pol η as described for human scFv library (see part I). PCR amplified VHH pool was digested with BglI and directionally cloned into the BglI sites of f3TR1 phage vector. Ligated DNAs were transformed into E. coli MC1061 by electroporation (160 transformations) to obtain a moderate size mutagenized f3TR1-VHH phage library containing ˜2.8×10⁸ independent clones. PCR analyses showed that over 90% of phage clones contain an insert consistent with VHH size (350-380 bp).

Materials. f3TR1 phage display vector and E. coli strain K91BK were obtained from George P. Smith (University of Missouri, Columbia, MO). Naïve VHH library was purchased from Abcore, Inc. (Ramona, CA). VHH repertoire in this library was prepared from Periferal Blood Mononuclear cells derived from 24 non-immunized Llamas and cloned into pADL20c phagemid vector.
cDNA preparation. Tonsils collected from tonsillectomy of 7 individuals were frozen in liquid nitrogen and pulverized using pestle and mortar. Total RNA were extracted using TRIzol reagent (ThermoFisher Scientific) according to the manufacturer protocol. ProtoScript II First Strand cDNA synthesis kit (New England Biolabs, MA) was used for cDNA preparation. 16 PCR tubes (40 μl volume each), containing 15 micrograms of RNA and the random primer mix (oligo-dT18 and random hexamers, 6 μM) was denatured at 65° C. for 5 min and placed on ice. After addition of the ProtoScript reaction mix and enzyme mix, cDNA synthesis was carried out by incubation 25° C. for 5 min followed by 42° C. for 60 min and enzyme inactivation at 80° C. for 5 min. cDNA were pooled and stored at −70° C.
PCR Amplification of variable regions of Ab heavy chains (VH) and light chain (VL) repertoire. In order to reduce amplification bias, 1^{st} PCR amplification was carried out in independent PCR tubes to amplify individual V gene segments, using all possible combinations with VH and VL forward and reverse primers. The primer sequences, allowing amplification of the entire repertoire of human antibody genes are listed in Table 3. Each PCR reaction (50 μl volume) was carried out in the presence of 375 ng of cDNA, 100 ng of each forward and reverse primers and 25 μl of 2×PCR master mix (Promega Corp.) using following PCR program: 94° C.—2 min; 94 oC—1 min, 55° C.—1 min, 72° C.—2 min for 30 cycles; 72° C. for 10 min. PCR products were separated by 1.2% TAE agarose gel electrophoresis and PCR bands corresponding to V genes were cut out (VH: ˜380 bp, kappa/lambda: ˜650 bp), purified by QIAquick Gel Extraction Kit (Qiagen) and eluted in 10 mM Tris, pH 8.5. PCR products from each subfamily (VH, kappa, lambda) were pooled separately and stored at −20° C.

The second round of PCR introduced BglI restriction sites at the 5′-end of VH and at the 3′-end of Vk and Vl genes. Reverse primers for VH and forward primers for Vk and Vl also contain additional sequences to form a flexible (G₄S)₄ (SEQ ID NO:1) linker between VH and VL to constitute scFv. The primers for 2nd PCR amplification are listed in Table 3. PCR reactions (50 μl volume) were set up for all pair of forward and reverse primers in the presence of 10-20 ng of a purified PCR product from the first PCR, 100 ng of each forward and reverse primers and 25 μl of 2×PCR master mix (Promega Corp.). PCR program is as follow: 94° C.—1 min; 94° C.—30 s, 55° C.—30 s, 72° C.—1 min for 25 cycles; 72° C. for 5 min. The 2^{nd} PCR amplification produced single VH or VL product bands (˜430-450 bp), which were combined for each subfamily and purified by Qiaquick PCR purification kit (Qiagen).

scFv diversification by AID and Pol η. VH, Vk and Vl PCR products were mixed together at molar ratios of 2 VH:1 Vk:1 Vl to constitute a V gene mixture. AID deamination was carried out in a series of PCR tubes using a thermocycler. Each tube, containing 200 ng of the V gene mix in H₂O (50 μl volume) was heat-denatured at 94° C. for 2 min and quickly cooled down to 37° C., followed by an immediate addition of 4 μl of pre-activated GST-AID (100 ng). Pre-activated AID was prepared by incubation of GST-AID (100 ng) and RNase (100 ng) for 3 min at 37° C. After incubation at 37° C. for predetermined time (30 s, 45 s, 1 min, 2 min, 5 min, 10 min or 20 min), AID deamination was stopped by twice extracting the reaction mixture with phenol:chlorophorm:isoamyl alcohol (25:24:1). Deaminated V gene DNAs were combined, desalted 3 times with H₂O and concentrated using Amicon Ultra-0.5 10 kDa centrifugal filter unit (Millipore).

Pol η error-prone synthesis was performed as follow. A PCR tube (50 μl volume), containing 50-100 ng of the V gene mix (AID-treated or non-AID treated), 10 ng of a forward and reverse primers (scFv-F: TACTCGCGGCCCACGCGGCCA (SEQ ID NO:6), scFv-R: TGGTGTTGGCCTCAGCGGCACT (SEQ gD NO:7)) in Pol η reaction buffer 40 mM Tris-HCl (pH 8.0), 50 mM NaCl, 2.500 glycerol, 10 mM Dithiothreitol, 2.5 mM MgCl₂ and 500 μM each of the four dNTPs was heat denatured and cooled to 4° C. to allow the annealing of the primers to V genes. Since the 3′-end of VH and the 5-end of Vk/Vl PCR products contain a complementary sequence (GGC GGC GGC GGC TCC (SEQ ID NO:8), annealed top strands of VH and bottom strands of Vk/Vl can also serve as primers for Pol η extension. DNA synthesis was initiated by addition of 300 ng of purified human Pol η and incubated for 2 h at 37° C. Synthesis reaction was terminated and Pol η was removed by twice extracting the reaction mixture with phenol:chlorophorm:isoamyl alcohol (25:24:1). Pol η-treated DNAs from 20-24 tubes were combined, desalted 4 times with H₂O using Amicon Ultra-0.5 10 kDa centrifugal filter unit (Millipore) and stored in 1 mM Tris (pH 8.0), 0.1 mM EDTA at −20° C.

TABLE 3
Primers used for the first PCR
amplification of human Ab heavy (VH)
and light (VL) chain repertoire.
Heavy chain VH 1^{st} PCR forward primers

| | |
|---|---|
| VH1-f | caggtbcagctggtgcagtctgg (SEQ ID NO: 46) |
| VH1/7-f | carrtscagctggtrcartctgg (SEQ ID NO: 47) |
| VH2-f | cagrtcaccttgaaggagtctgg (SEQ ID NO: 48) |
| VH3-f1 | sargtgcagctggtggagtctgg (SEQ ID NO: 49) |
| VH3-f2 | gaggtgcagctgktggagwcysg (SEQ ID NO: 50) |
| VH4-f1 | caggtgcarctgcaggagtcggg (SEQ ID NO: 51) |
| VH4-f2 | cagstgcagctrcagsagtssgg (SEQ ID NO: 52) |
| VH5-f | gargtgcagctggtgcagtctgg (SEQ ID NO: 53) |
| VH6-f | caggtacagctgcagcagtcagg (SEQ ID NO: 54) |

Heavy chain VH 1^{st} PCR reverse primers

| | |
|---|---|
| IgMCH1-r | aagggttggggcggatgcact (SEQ ID NO: 55) |
| IgGCH1-r | gaccgatgggcccttggtgga (SEQ ID NO: 56) |

Kappa light chain 1^{st} PCR forward primers

| | |
|---|---|
| VK1-f1 | gacatccagatgacccagtctcc (SEQ ID NO: 57) |
| VK1-f2 | gmcatccrgwtgacccagtctcc (SEQ ID NO: 58) |
| VK2-f | gatrttgtgatgacycagwctcc (SEQ ID NO: 59) |
| VK3-f | gaaatwgtgwtgacrcagtctcc (SEQ ID NO: 60) |
| VK4-f | gacatcgtgatgacccagtctcc (SEQ ID NO: 61) |
| VK5-f | gaaacgacactcacgcagtctcc (SEQ ID NO: 62) |
| VK6-f | gawrttgtgmtgacwcagtctcc (SEQ ID NO: 63) |

Kappa light chain 1^{st} PCR reverse primer

| | |
|---|---|
| kappa-r | acactctcccctgttgaagctctt (SEQ ID NO: 64) |

Lambda light chain 1^{st} PCR forward primers

| | |
|---|---|
| VL1-f1 | cagtctgtgctgactcagccacc (SEQ ID NO: 65) |
| VL1-f2 | cagtctgtgytgacgcagccgcc (SEQ ID NO: 66) |
| VL2-f | cagtctgccctgactcagcct (SEQ ID NO: 67) |
| VL3-f1 | tcctatgwgctgacwcagccacc (SEQ ID NO: 68) |
| VL3-f2 | tottctgagctgactcaggaccc (SEQ ID NO: 69) |
| VL4-f1 | ctgcctgtgctgactcagccc (SEQ ID NO: 70) |
| VL4-f2 | cagcytgtgctgactcaatcryc (SEQ ID NO: 71) |
| VL5-f | cagsctgtgctgactcagcc (SEQ ID NO: 72) |
| VL6-f | aattttatgctgactcagcccca (SEQ ID NO: 73) |
| VL7/8-f | cagrctgtggtgacycaggagcc (SEQ ID NO: 74) |
| VL9/10-f | cagscwgkgctgactcagccacc (SEQ ID NO: 75) |

Lambda light chain 1^{st} PCR forward primers

| | |
|---|---|
| lambda-r1 | tgaacattctgtaggggccactg (SEQ ID NO: 76) |
| lambda-r2 | tgaacattccgtaggggcaactg (SEQ ID NO: 77) |

TABLE 4
Primers used for the second PCR amplification of human Ab
heavy (VH) and light (VL) chain repertoire.
Heavy chain VH 2^{nd} PCR forward primers*

| | |
|---|---|
| F3VH1fN | TACTCGCGGCCCACGCGGCCATGGCTcaggtbcagctggtgcagtctgg (SEQ ID NO: 78) |
| F3VH1/7fN | TACTCGCGGCCCACGCGGCCATGGCTcarrtscagctggtrcartctgg (SEQ ID NO: 79) |
| F3VH2fN | TACTCGCGGCCCACGCGGCCATGGCTcagrtcaccttgaaggagtctgg (SEQ ID NO: 80) |
| F3VH3f1N | TACTCGCGGCCCACGCGGCCATGGCTsargtgcagctggtggagtctgg (SEQ ID NO: 81) |
| F3VH3f2N | TACTCGCGGCCCACGCGGCCATGGCTgaggtgcagctgktggagwcysg (SEQ ID NO: 82) |
| F3VH4f1N | TACTCGCGGCCCACGCGGCCATGGCTcaggtgcarctgcaggagtcggg (SEQ ID NO: 83) |
| F3VH4fN | TACTCGCGGCCCACGCGGCCATGGCTcagstgcagctrcagsagtssgg (SEQ ID NO: 84) |
| F3VH5fN | TACTCGCGGCCCACGCGGCCATGGCTgargtgcagctggtgcagtctgg (SEQ ID NO: 85) |
| F3VH6fN | TACTCGCGGCCCACGCGGCCATGGCTcaggtacagctgcagcagtcagg (SEQ ID NO: 86) |

Heavy chain VH PCR reverse primers

| | |
|---|---|
| IgMscFVR | GGAGCCGCCGCCGCCAGAACCACCACCACCAGAACCACCACCACCggttggggoggatgcact |
| | (SEQ ID NO: 87) |
| IgGscFvR | GGAGCCGCCGCCGCCAGAACCACCACCACCAGAACCACCACCACCgaccgatgggcccttggtgga |
| | (SEQ ID NO: 88) |

Kappa light chain 2^{nd} PCR forward primers

| | |
|---|---|
| VK1Linkf1 | GGCGGCGGCGGCTCCGGTGGTGGTGGATCCgacatccagatgacccagtctcc (SEQ ID NO: 89) |
| VK1Linkf2 | GGCGGCGGCGGCTCCGGTGGTGGTGGATCCgmcatccrgwtgacccagtctcc (SEQ ID NO: 90) |
| VK2Linkf | GGCGGCGGCGGCTCCGGTGGTGGTGGATCCgatrttgtgatgacycagwctcc (SEQ ID NO: 91) |
| VK3Linkf | GGCGGCGGCGGCTCCGGTGGTGGTGGATCCgaaatwgtgwtgacrcagtctcc (SEQ ID NO: 92) |
| VK4Linkf | GGCGGCGGCGGCTCCGGTGGTGGTGGATCCgacatcgtgatgacccagtctcc (SEQ ID NO: 93) |
| VK5Linkf | GGCGGCGGCGGCTCCGGTGGTGGTGGATCCgaaacgacactcacgcagtctcc (SEQ ID NO: 94) |
| VK6Linkf | GGCGGCGGCGGCTCCGGTGGTGGTGGATCCgawrttgtgmtgacwcagtctcc (SEQ ID NO: 95) |

Kappa light chain 2^{nd} PCR reverse primer

| | |
|---|---|
| F3scFvkapR | TGGTGTTGGCCTCAGCGGCACTAGTgaagacagatggtgcagccacagt (SEQ ID NO: 96) |

Lambda light chain 2^{nd} PCR forward primers

| | |
|---|---|
| VL1Linkf1 | GGCGGCGGCGGCTCCGGTGGTGGTGGATCCcagtctgtgctgactcagccacc (SEQ ID NO: 97) |
| VL1Linkf2 | GGCGGCGGCGGCTCCGGTGGTGGTGGATCCcagtctgtgytgacgcagccgcc (SEQ ID NO: 98) |
| VL2Linkf | GGCGGCGGCGGCTCCGGTGGTGGTGGATCCcagtctgccctgactcagcct (SEQ ID NO: 99) |
| VL3Linkf1 | GGCGGCGGCGGCTCCGGTGGTGGTGGATCCtcctatgwgctgacwcagccacc (SEQ ID NO: 100) |
| VL3Linkf2 | GGCGGCGGCGGCTCCGGTGGTGGTGGATCCtcttctgagctgactcaggaccc (SEQ ID NO: 101) |
| VL4Linkf1 | GGCGGCGGCGGCTCCGGTGGTGGTGGATCCctgcctgtgctgactcagccc (SEQ ID NO: 102) |
| VL4Linkf2 | GGCGGCGGCGGCTCCGGTGGTGGTGGATCCcagcytgtgctgactcaatcryc (SEQ ID NO: 103) |
| VL5Linkf | GGCGGCGGCGGCTCCGGTGGTGGTGGATCCcagsctgtgctgactcagcc (SEQ ID NO: 104) |
| VL6Linkf | GGCGGCGGCGGCTCCGGTGGTGGTGGATCCaattttatgctgactcagcccca (SEQ ID NO: 105) |
| VL7/8Linkf | GGCGGCGGCGGCTCCGGTGGTGGTGGATCCcagrctgtggtgacycaggagcc (SEQ ID NO: 106) |
| VL9/10Linkf | GGCGGCGGCGGCTCCGGTGGTGGTGGATCCcagscwgkgctgactcagccacc (SEQ ID NO: 107) |

Lambda light chain 2^{nd} PCR reverse primers

| | |
|---|---|
| F3scFvlamR | TGGTGTTGGCCTCAGCGGCACTAGTagaggasggygggaacagagtgac (SEQ ID NO: 108) |

*Bases in caps font are for BglI sites and formation of the (G₄S)₄ (SEQ ID NO: 1) linker

Generation of scFv repertoire by overlapping PCR. V genes treated with both AID and Pol η was combined with V genes treated with AID alone and V genes treated with Pol η alone and non-treated V genes at an equal concentration to use as templates to generate mutagenized scFv repertoire. The 3′-end of VH and the 5-end of Vk/Vl contain complementary sequences allowing a fusion of VH and VL and forming a (G4S)4 (SEQ ID NO:1) linker for scFv by overlapping PCR. 96 PCR tubes (50 μl volume), each contains 10 ng of the V gene mixture, 100 ng of a forward primer (scFv-F: TACTCGCGGCCCACGCGGCCA (SEQ ID NO:6)), 100 ng of reverse primer (scFv-R: TGGTGTTGGCCTCAGCGGCACT (SEQ ID NO:7)) and 25 μl of 2×PCR master mix (Promega) were subjected to 30 cycles of PCR: 94° C.—1 min; 94° C.—30 s, 55° C.—30 s, 72° C.—1 min for 30 cycles; 72° C. for 2 min. Overlapping PCR produced a single PCR product (˜850-900 bp) corresponding to scFv composition VH-(G₄S)₄ (SEQ ID NO:1)-VL. PCR products were combined, extracted twice with phenol:chlorophorm:isoamyl alcohol (25:24:1) and ethanol precipitated. The mutagenized scFv gene repertoire was resuspended in 10 mM (Tris pH 8.5) and stored at −20° C.
Cloning of scFv into f3TR1 phage display vector and generation of f3TR1-scFv phage display library. The mutagenized scFv gene repertoire and f3TR1 vector dsDNA were digested with BglI (New England Biolabs) for 3 h at 37° C. Digested scFv DNA were purified using QIAquick PCR purification kit (Qiagen) and eluted in 10 mM Tris (pH 8.5). The digested f3TR1 was de-phosphorylated with Shrimp Alkaline Phosphatase (New England Biolabs), purified using a Qiagen-tip 500 column (Qiagen) and resuspended in 10 mM Tris (pH 8.5). Ligation of scFv into f3TR1 vector was carried out at 16° C. for 16 h by T4 DNA ligase (New England Biolabs) using scFv:f3TR1 molar ratio of ˜2:1. Ligated DNAs were desalted 4 times with H₂O and concentrated using Amicon Ultra-15 10 kDa centrifugal unit (Millipore) and stored at −20° C.

The ligated DNAs were electroporated into E. coli competent cells MC1061 using BioRad electroporator. To generate the mutagenized scFv library, a total of 240 electroporation was carried out, in a batch of 20 cuvettes. About 50 ng of ligated DNA was used for a transformation of 50 μl of the competent cells. After electroporation each cuvette was flushed with 1 ml of SOC medium and transformed cells from all 20 cuvettes were combined in a 100 ml flask. The flask was incubated at 37° C. in a New Brunswick shaker (200 rpm) for 30-45 min and the entire content was transferred into a prewarmed 2 L flask containing 1 L of 2×YT medium supplemented with 20 μg/ml tetracycline. Aliquots (10 μl to 200 μl) from the flask with 2×YT medium were plated immediately on LB plates containing tetracycline (20 μg/ml). After incubation overnight at 37° C., the number of colonies was used to determine the number of independent transformants for each batch. Numbers of independent transformants from all 12 electroporation batches were combined to calculate the size of the mutagenized scFv library (˜1.1×10⁹).

Purification of primary f3TR1-scFv phage library. Flasks with transformants in 2×YT medium were incubated at 37° C. for 16 h to allow the production of primary f3TR1-scFv phages. The culture supernatants (total of 12 L) were collected after centrifugation and phages were precipitated by adding 15% volume of PEG/NaCl solution (16.6% PEG 8000 MW, 3.3 M NaCl) for 4 h at 4° C. Precipitated phages were harvested by centrifugation at 10,000×g for 20 min. The phage pellets were resuspended in TBS buffer (1/30 volume of supernatant) and subjected to a second round of PEG/NaCl precipitation. Phage pellet from the second precipitation was resuspended in TBS buffer and 50% glycerol (typically 1 ml for each liter of supernatant) and stored at −20° C.
Titration of f3TR1-scFv phage tetracycline transducing units (TU). Aliquots (10 μl each) of serially diluted phage library (10⁻², 10⁻⁴, 10⁻⁶, 10⁻⁸, 10⁻¹⁰ dilution in TBS buffer) were mixed with 0.5 ml of mid-log K91BK cells in 2×YT medium and incubated at 37° C. to allow infection. After 30 min, infected cells (100 μl) were plated on LB plates supplemented tetracycline (20 μg/ml) and kanamycin (20 μg/ml). After incubation at 37° C. overnight, phage library titer of transducing unit (TU) was calculated based on numbers of colonies on plates and dilution factor.
Preparation of high-titer phage stocks for bio-panning. K91BK cells were grown in 1 L flask containing 500 ml of 2×YT at 37° C. until OD₆₀₀ of 1/10 dilution is 0.2 (˜2.5×10¹² cells) and infected with primary scFv phage (7.5×10¹¹ TU). The flask was incubated without shaking for 15 min and six 90 ml each of the culture were distributed to six flasks each contains 1 L of prewarmed 2×YT medium supplemented with 0.22 μg/ml tetracycline (6 flasks total). After shaking at 37° C. for 35 min, tetracycline was added to a final concentration of (20 μg/ml), and phage production was continued for 16 h. A high titer human scFv phage library stock (5×10¹² TU/ml) was harvested and purified as described above.
Generation and preparation of mutagenized Llama f3TR1-VHH phage library. Llama VHH repertoire was PCR amplified from Abcore's naïve VHH library using a forward (TATTACTCGCGGCCCACGCGGCCATGGCT (SEQ ID NO:9)) and a reverse (GGTGATGGTGTTGGCCCCAGGGGCTGAGGAGACGGTGAC (SEQ ID NO:10)) primers, which incorporate BglI restriction sites for cloning into f3TR1. VHH repertoire PCR products were subjected to AID and Pol η diversification using procedures described for the mutagenized human scFv library. 160 transformations by electroporation were carried out to obtain a mutagenized Llama VHH library (2.8×10⁸ independent clones). A high titer VHH phage library stock (1.9×10¹³ TU/ml) was prepared for bio-panning.
Phage library clone analysis. Randomly selected K91BK colonies on LB+tetracycline plates from titration of primary mutagenized phage-scFv and phage-VHH libraries were picked and resuspended in 50 μl of sterile H₂O. Resuspended cells were lysed by incubation at 99° C. for 5 min and 2 μl of the lysate supernatant was added to a PCR tube (25 μl final volume) containing 12.5 μl of 2×PCR master mix (Promega), 50 ng each of SF3-F forward (TTTGGAGATTTTCAACGTTGA (SEQ ID NO:11)) and SF3-F reverse (GCGTAACGATCTAAAGTTTTGTCG (SEQ ID NO:12)) primers. After 30 cycles (94° C.—1 min, 50° C.—30 s, 72° C.—2 min), 5 μl of PCR product was analyzed by 1.2% agarose gel electrophoresis. For DNA sequencing, PCR product was treated with ExoProStar sequencing reaction clean-up kit (GE healthcare) and entire scFv or VHH was subjected to Sanger sequencing from both directions using SF3-F and PsiR3 as sequencing primers. VH and VL chains of sequenced scFv clones were analyzed using IgBLAST tool.

### Example 3. Application of AID and Pol η-Affinity Maturation In Vitro to Isolate Antibodies Targeting Specific Antigens

We have applied the mutagenized human scFv and Llama VHH libraries and in vitro affinity maturation to isolate antigen-specific Abs targeting human Glucagon-like peptide-1 receptor (GLP-1R), human fatty acid amide hydrolase (FAAH), mouse artemin, and human AID variant protein (AIDv).

### I. Glucagon-Like Peptide-1 Receptor (GLP-1R)

The human mutagenized scFv library was used in 4 rounds of panning using GLP-1R-expressing Sf9 cells as the target antigens. The phage outputs from the 3^{rd} and 4^{th} panning were used to prepare scFv gapped DNA and subjected to AID and Pol η diversification as described in Example 1. The resulting scFv sub-library was applied to 2 rounds of panning on a purified His₆ (SEQ ID NO:13)-tagged GLP-1R protein. Eight scFv clones were isolated and their binding to the purified GLP-1R was verified by ELISA. Gapped DNA constructs prepared from the eight isolated scFv clones were mutated by AID and Pol η and transformed into E. coli to obtain a pool of mutagenized scFv phage. The pool was used in 3 rounds of panning on the purified GLP-1R protein. One mutated scFv clone (GHM33) that has the highest affinity was isolated. Measurement of binding constant Kd by titration ELISA showed that the purified GHM33 scFv protein apparent dissociation constant (K_{d}=100 nM) is ˜3-fold lower than the parental scFv (K_{d}=320 nM). Thus, one round of in vitro affinity maturation of anti-GLP-1R Abs lead to a 3-fold improvement in binding affinity.

The DNA sequence of VH in clone GHM33:
(SEQ ID NO: 111)
GTGCAGCTGGTGCAGTCTGGGGCTGAGGTGAAGAAGCCTGGGTCCTCGGT
GAAGGTCTCCTGCAAGGCTTCTGGAGGCACCTTCAGCAGCTATGCTATCA
GCTGGGTGCGACAGGCCCCTGGACAAGGGCTTGAGTGGATGGGAGGGATC
ATCCCTATCTTTGGTACAGCAAACTACGCACAGAAGTTCCAGGGCAGAGT
CACGATTACCGCGGACGAATCCACGAGCACAGCCTACATGGAGCTGAGCA
GCCTGAGATCTGAGGACACGGCCGTGTATTACTGTGCGAGGGGTCGGGAT
GATTACTATGATAGTAGTGCCTTTGACTACTGGGGCCAGGGAACCCTGGT
CACCGTC.
The peptide sequence of VH in clone GHM33:
(SEQ ID NO: 112)
VQLVQSGAEVKKPGSSVKVSCKASGGTFSSYAISWVRQAPGQGLEWMGGI
IPIFGTANYAQKFQGRVTITADESTSTAYMELSSLRSEDTAVYYCARGRD
DYYDSSAFDYWGQGTLVTV.
The DNA sequence of VL in clone GHM33:
(SEQ ID NO: 113)
GTGCTGACTCAGCCACCCTCGGTGTCAGTGGCCCCAGGACAGACGGCCAG
GGTTACCTGTGGGGGAAACAAGATTGGAAGTTACAGTGTCTACTGGTACC
AGCACAAGCCTGGCCAGGCCCCTGTGTTGGTCGTCTATGATGATAGTGAC
CGGCCCTCAGGGATCCCTGAACGATTCTCTGGCTCCAACTCTGGGAACAT
GGCCACCCTGACCATCAGCGGGGTCGAAGCCGGGGATGAGGCCGACTATT
ACTGTCAGGTGTGGGATTGTACTACTGATGATGTGATCTTCGGCGGAGGG
ACCAAGCTGACCGTCCTA.
The peptide sequence of VL in clone GHM33:
(SEQ ID NO: 114)
VLTQPPSVSVAPGQTARVTCGGNKIGSYSVYWYQHKPGQAPVLVVYDDSD
RPSGIPERFSGSNSGNMATLTISGVEAGDEADYYCQVWDCTTDDVIFGGG
TKLTVL.

### II. Fatty Acid Amide Hydrolase (FAAH)

The Llama mutagenized VHH library was applied to 3 rounds of panning against a purified human FAAH protein. For each round of panning, VHH phages were pre-incubated a purified MBP protein immobilized on the tube surface to remove phages specific to the MBP-tag. One clone (A3) with the highest ELISA signal was selected and its VHH sequence was subjected AID and pol η mutagenesis. Five VHH clones were isolated after 1 round of AID and Pol η affinity maturation in vitro on clone A3. One mutated VHH clone (OD1) with higher affinity was isolated. Measurement of binding kinetics of purified VHH nanobodies by surface plasmon resonance (SPR) showed that affinity-matured OD1 VHH nanobody has ˜2.4-fold increase in FAAH affinity (Kd=0.65 nM) compared to parent VHH A3 nanobody (Kd=1.56 nM).

The DNA sequence of VHH in clone OD1:
(SEQ ID NO: 115)
GTGCAGCTTGTGGAGTCTGGGGGAGGCTTGGTGCAGGCTGGGGGGTCTCT
GAGACTCTCCTGTGCAGCCTCTGGACGAACCTTCAGTGACAACACCATGG
CCTGGCACCGCCAGGGTCCAGGGAAGCAGCGCGGGTTGGTCGCATTTATC
ACAACTGGTGGCCGTACAGTCTATACCGACTCCGTGAAGGGCCGATTCAT
CATCTCCAGAGACAACGCCAAGAACACGGGGTTTCTGCAAATGAACAGCC
TGAAACCTGAGGACACGGCCGTCTATTACTGTTATGTAGCCGGTAATTGG
GGCCAGGGGACCCAGGTCACCGTC.
The peptide sequence of VHH in clone OD1:
(SEQ ID NO: 116)
VQLVESGGGLVQAGGSLRLSCAASGRTFSDNTMAWHRQGPGKQRGLVAFI
TTGGRTVYTDSVKGRFIISRDNAKNTGFLQMNSLKPEDTAVYYCYVAGNW
GQGTQVTV.

### III. Recombinant Mouse Artemin Protein

Llama mutagenized and naïve VHH libraries were used in parallel to select for nanobodies that bind to a purified mouse artemin (Ala112-Gly224 fragment). After 3 rounds of panning and sequence analysis of isolated clones, 2 pairs of related nanobodies in mutagenized and naïve libraries were identified. Two clones LM41 and LM52 from the VHH mutagenized library likely represent AID and Pol η-induced mutant variants of LU68 and LU5 clones from the naïve VHH library. SPR measurement showed that the isolated mutated nanobodies have a higher binding, compared to the corresponding original nanobodies (Kd=111 nM for LM41 vs Kd=456 nM for LU68 and Kd=102 nM for LM52 vs Kd=178 nM for LU5).

The DNA sequence of VHH in clone LM52:
(SEQ ID NO: 117)
GTGCAGCTGGTAGAGTCTGGGGGAGGATTGGTGCAGGCTGGGGCCTCTCT
GAGACTCTCCTGTGCAGCCTCTGGAGGTACTCTCGTCAACTCTAGTATGG
GCTGGTTCCGCCAGGCTCCAGGGAAGGAGCGTGACTTCGTGGCAACTATT
AACTGGCGCGGTGATACTACATGGTATGGAGAGTCCGTGAAGGGCCGATT
CTCCATCTCCAGAGACAACACCAAAAACATGGTGTACCTGCAAATGAACA
GCCTGAAACCTGAGGACACGGCCGTTTATTACTGTGCAGTCTACAGGACA
AGATACTATACTGGCCGTCGCATGATGTCCCCAGATGAGTATGACGTTTG
GGGCCAGGGGACCCAGGTCACCGTC.
The peptide sequence of VHH in clone LM52:
(SEQ ID NO: 118)
VQLVESGGGLVQAGASLRLSCAASGGTLVNSSMGWFRQAPGKERDFVATI
NWRGDTTWYGESVKGRFSISRDNTKNMVYLQMNSLKPEDTAVYYCAVYRT
RYYTGRRMMSPDEYDVWGQGTQVTV.

### IV. Human AID Variant Protein (AIDv)

The mutagenized human scFv and Llama VHH libraries were used to screen for Abs targeting AIDv protein. Three rounds of panning were performed targeting a purified E. coli expressed MBP-AIDv followed by an additional final round of panning on a Sf9-expressed untagged AIDv protein. Five human scFv and three Llama VHH clones were isolated. Binding of scFv and VHH Abs to the purified AIDv was verified by ELISA.

The DNA sequence of VH in clone 3:
(SEQ ID NO: 119)
GTGCAGCTGGTGCAGTCTGGGGGAGGCTTGGTACAGCCTGGGGGGTCTCT
GCGACTCTCCTGTGTAGCCTCTGGATTCACCTTTACCGACTATGCCGTGA
CCTGGGTCCGCCAGGCTCCAGGGAAGGGTCTGGAGTGGGTCTCAGCTATC
AGTGGTAGTGGTGGAAACACATACTACGCAGACTCCGTGAAGGGCCGGTT
CACCGTCTCCAGGGACAATTCCGACAACACGGTGTCTCTGCAAATGAACA
GCCTGAGAGTCGAGGACACGGGCATATATTATTGTGCGAAGTCGGCTCGG
ATCTTAAATGGATATTACTTCTATGCTATGGACGTCTGGGGCCAAGGGAC
CACGGTCACCGTC.
The peptide sequence of VH in clone 3:
(SEQ ID NO: 120)
VQLVQSGGGLVQPGGSLRLSCVASGFTFTDYAVTWVRQAPGKGLEWVSAI
SGSGGNTYYADSVKGRFTVSRDNSDNTVSLQMNSLRVEDTGIYYCAKSAR
ILNGYYFYAMDVWGQGTTVTV.
The DNA sequence of VL in clone 3:
(SEQ ID NO: 121)
CAGTCTGCCCTGACTCAGCCTGCCTCCGTGTCTGGGTCTCCTGGACAGTC
GATCACCGTCTCCTGCACTGGAACCAGCAGTGATGTTGCGAAATATAACC
TTGTCTCCTGGTACCAACAGCACCCAGGCAAAGCCCCCGAACTCATGATT
TATGATGTCAGTAAGCGGCCCTCAGGGGTCCCTGATCGCTTCTCTGGCTC
CAAGTCTGGCAACACGGCCTCCCTGACCATTTCTGGGCTCCAGCCTGACG
ATGAGGCTTATTATCACTGCAGCTCATATGGAGGCACCAACAATTTGCTA
TTCGGCGGAGGGACCAAGGTGACCGTTCTA
The peptide sequence of VL in clone 3:
(SEQ ID NO: 122)
QSALTQPASVSGSPGQSITVSCTGTSSDVAKYNLVSWYQQHPGKAPELMI
YDVSKRPSGVPDRFSGSKSGNTASLTISGLQPDDEAYYHCSSYGGTNNLL
FGGGTKVTVL.

Antigens. Purified recombinant mouse artemin protein (Ala112-Gly224 fragment) purchased as lyophilized powder from R&D Systems, Inc., was reconstituted at 100 mg/mL in 4 mM HCl and stored at −20° C. E. coli expressed MBP-AIDv and Sf9-expressed untagged AIDv proteins were purified as described. Live cultures of Sf9 cells expressing a recombinant human His₆-tagged Glucagon-like peptide-1 receptor (GLP-1R) and biomass for purification were obtained from Raymond Stevens' lab (Bridge Institute, University of Southern California, CA). Recombinant GLP-1R protein was purified as described. Human fatty acid amide hydrolase, FAAH (accession AH007340.2) was expressed and purified as follow. FAAH sequenced corresponding to amino acids 30-579 (without the N-terminal transmembrane domain) was cloned into pMALx expression vector and expressed in E. coli CSH50 cells at 18° C. overnight as MBP-tag fusion protein. The cells were harvested by centrifugation and washed once with 10 mM Tris pH 8.5+1M NaCl. The pellet was resuspended in lysis buffer 20 mM Tris pH 8.5, 0.1% Triton X-100, 5 mM DTT, 1 M NaCl, 1 mM PMSF and lysed by sonication. After centrifugation at 20,000×g for 30 min, the supernatant was incubated with Amylose resin (New England Biolabs). MBP-FAAH fusion protein was eluted with 40 mM Maltose and further purified by gel filtration using Superdex 200 gel filtration column (GE Healthcare). To purify untagged FAAH, the MBP tag was cleaved with Factor Xa Protease for 3 hours in buffer containing 20 mM Tris-HCl pH 8.5, 100 mM NaCL, 2 mM CaCl₂. Untagged FAAH protein was further cleaned by gel filtration. MBP-FAAH and FAAH proteins were stored in 20 mM Tris-HCl pH 8.5, 1M NaCl and 5% Glycerol at −80° C.

### Selection of Phage Antibody Library by Bio-Panning

Enrichment of phage particles displaying specific human scFv or Llama VHH were performed on MaxiSorp Nunc-Immunotubes. Protein antigens (3-10 μg/ml) in 1 ml of phosphate-buffered saline (PBS, pH 7.4) (50 mM Sodium bicarbonate pH 9.6 in case of artemin) were coated on the tube surface overnight at 4° C. After blocking with 2% (w/v) skimmed milk powder in PBS (2% MPBS) for 1-2 h, an aliquot of phage library (f3TR1-scFv or f3 TR1-VHH) containing 1×10¹² phage TU was added to the tube in the 1^{st} round of panning. For the subsequent rounds of panning, less phages (2×10¹¹ TU) were used. After gentle rocking for 2 hours at room temperature. Non-bound phages were eliminated by washing 10-15 times with PBS containing 0.1% Tween 20 (PBS-T), followed by 2 times washing with PBS. The bound phages were eluted by incubation with 1 ml of 10 μg/ml trypsin in PBS for 30 min at RT. Amplification of eluted phages was carried out by incubating 0.5 ml of eluted phage with 25 ml of exponentially growing E. coli K91BK cells in 2×YT medium without shaking for 30 min at 37° C. 75 ml of 2×YT medium supplemented with tetracycline (0.22 μg/ml) was added and the culture was transferred to a 37° C. shaker (200 rpm). After 45 min, tetracycline was added to 20 μg/ml and phage production was continued for 16 h. Titers of eluted phage binders and amplified phage preps for each panning were determined by serial dilution and infection of K91BK cells as described for phage library titration.

Sf9 cell panning was carried out at 4° C., scFv phage (1×10¹² TU) was pre-incubated with 2×10⁷ control Sf9 cells in PBS+2.5% BSA for 1-2 h to deplete non-specific binding phages. 1×10⁷ GLP-1R expressing cells were blocked in PBS+2.5% BSA for 1 h and incubated with depleted phages for 1 h. Ice cold PBS was used for washing (10 times) by centrifugation at 300×g for 5 min. Bound phages were eluted by resuspending cells in 500 μl of PBS containing 10 μg/ml trypsin and incubate at RT for 30 min.

Screening for antigen-specific clones by phage ELISA. Individual K91BK colonies on titration plates from the last panning were pick into 96-deep well plates containing 1.5 ml of 2×YT+20 μg/ml tetracycline and grown overnight in a shaker at 37° C. to produce scFv or VHH phage particles. Phages from 1 ml of supernatant were precipitated with 150 μl of the PEG/NaCl solution and resuspended in 200 μl of PBS.

Each well of Nunc Maxisorp 96-well microplates was coated with 100 μl of 5-10 μg/ml of each antigen. After overnight incubation at 4° C., plates were blocked with 2% MPBS for 1.5 hour at RT followed by 3 washed with PBS-T and 3 washes with PBS. All washings were performed on AquaMax 2000 plate washer (Molecular Devices, LLC). The selected phage preparation was diluted 1:2 in 4% MPBS before adding 100 μl into each well, and incubated for 1.5 hour at RT. The plates were washed three times with PBS-T, followed by three times with PBS, and incubated with 100 μl of a 1:4000 dilution of anti-M13-HRP (Sino Biological US, Inc., Wayne, PA) in 2% MPBS for 1 h at RT. Plates were washed 4 times with PBS-T and 4 times with PBS. TMB substrate solution 100 μl (Thermo Fisher Scientific) was added to each well (for 2 min to 30 min). After adding 100 μl of 2 M sulfuric acid stop solution, the absorbance was read at 450 nm, using SpectraMax iD5 microplate reader (Molecular Devices, LLC).

Expression and purification of soluble scFv and VHH Abs. Coding sequences for antigen-specific scFv and VHH Abs were subcloned into the BglI sites of pADL-20c phagemid vector. The constructs were transformed into E. coli CSH50 cells and Abs were expressed as His₆-tagged soluble proteins in the periplasm. Cells were grown at 37° C. in LB+0.2% glucose+Ampicillin (100 μg/ml). When the culture OD₆₀₀ reaches ˜0.7, IPTG (1 mM) was added to induce scFv/VHH expression at 30° C. overnight. Cells from 1 L culture were harvested by centrifugation, resuspended in 400 ml ice-cold wash buffer (30 mM Tris pH 8.0 and 20% glucose and EDTA was added to 1 mM. After incubation at 4° C. for 10 min, cells were spun down at 8000×g for 20 min. The pellet was resuspended in 400 ml of ice cold 5 mM MgSO₄ and incubated for 10-15 min at 4° C. The supernatant containing a soluble scFv/VHH protein was collected by centrifugation at 8000×g for 20 min. His₆ (SEQ ID NO:13)-tagged scFv/VHH proteins were purified by affinity chromatography using 2-3 ml of Ni-NTA resin (Qiagen) using PBS as a wash buffer and eluted in PBS containing 300 mM Imidazole. In some cases, scFv/VHH were further purified by gel filtration using Superdex 75 column (GE Healthcare). Proteins were concentrated using Amicon Ultra-15 10 kDa centrifugal filter unit (Millipore) and stored in PBS at −70° C.
Titration ELISA. Titration ELISA was used to measure binding of purified scFv to GLP-1R. GLP-1R immobilization in a 96 well plate and blocking was carried out as described for phage ELISA. A purified scFv was prepared at varying concentrations (from 1 nM to 3 μM) in 2% MPBS. 100 μl scFv aliquot of each concentration was added to a well and incubated at RT for 1.5 h. The plate was then washed with TPBS and PBS three times each. Primary anti His-tag Mouse mAb (Cell signaling Technology, Inc.) was diluted 1:250 in 2% MPBS and added into each well. After 1.5 incubation, wells were washed and secondary anti-mouse (goat-anti mouse-HRP; Santa Cruz Biotechnology) diluted 1:1000 in 2% MPBS was added and incubated for 1 h. After washing, TMB substrate solution was added for color development (15 to 30 min) and 2M sulfuric acid was added to stop the reaction. The absorbance was read at 450 nm, using SpectraMax iD5 microplate reader (Molecular Devices, LLC). The dissociation constant Kd was calculated according to a published protocol.
Surface plasmon resonance (SPR). Binding of purified VHH nanobodies to Artemin and to untagged FAAH proteins was determined using Biacore T100 instrument (GE Healthcare). Either the target antigen or individual VHH nanobody was suspended in 10 mM sodium acetate (pH 4.5) and immobilized on a CM5 Series S sensor chip (GE Healthcare) at 150-250 RU (response unit) using amine coupling chemistry according to the manufacturer's protocol (GE Healthcare). VHH Abs or target antigen at a concentration ranging from 10-500 nM in flow buffer (PBS-0.005% Tween) was injected onto the flow cells (flow rate 30 ml/min) for 120 seconds. The sensor chip surface was regenerated using 4 mM NaOH or 7 mM NaOH. Kinetic constants for binding interaction were determined by fitting the sensorgrams with 1:1 binding model using Biacore T100 evaluation software, version 2.0 (GE healthcare).

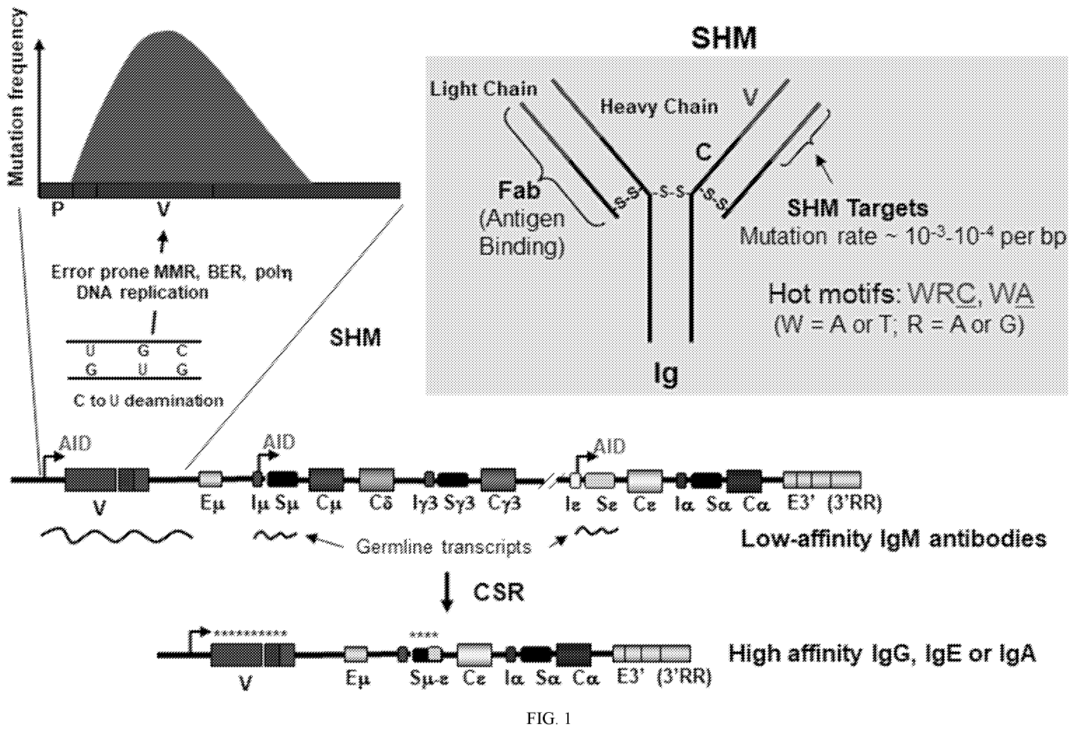

Without wishing to be bound by a particular theory, a general mechanism CSR is as follows. In humans, the order of the heavy chain exons is generally as follows: μ—IgM, δ—IgD, γ3—IgG3, γ1—IgG1, α1—IgA1, γ2—IgG2, γ4—IgG4, ε—IgE, and α2—IgA2. During CSR, portions of the antibody heavy chain locus are removed from the chromosome, and the gene segments surrounding the deleted portion are rejoined to retain a functional antibody gene that produces antibody of a different isotype. Double-stranded breaks are generated in DNA at conserved nucleotide motifs, called switch (S) regions, which are upstream from gene segments that encode the constant regions of antibody heavy chains; these occur adjacent to all heavy chain constant region genes with the exception of the δ-chain. DNA segment is removed by enzyme activity between switch regions. That is, DNA is nicked and broken at two selected S-regions by the activity of a series of enzymes, including activation-induced (cytidine) deaminase (AID), uracil DNA glycosylase and apyrimidic/apurinic (AP)-endonucleases; and the intervening DNA between the S-regions is subsequently deleted from the chromosome, removing unwanted μ or δ heavy chain constant region exons and allowing substitution of a γ, α or ε constant region gene segment (FIG. 1). The free ends of the DNA are rejoined by a process called non-homologous end joining (NHEJ) to link the variable domain exon to the desired downstream constant domain exon of the antibody heavy chain.

V(D)J recombination is the mechanism of somatic recombination that occurs in developing lymphocytes during the early stages of T and B cell maturation. Each heavy chain or light chain gene contains (1) multiple copies of three different types of gene segments (V, J, D) for the variable regions of the antibody proteins, and (2) one or more Constant gene segments for the constant regions. For example, the human immunoglobulin heavy chain region contains 2 Constant (Cμ and Cδ) gene segments and 44 Variable (V) gene segments, plus 27 Diversity (D) gene segments and 6 Joining (J) gene segments. The light chain genes possess either a single (Cκ) or four (Cλ) Constant gene segments with numerous V and J gene segments but do not have D gene segments. DNA rearrangement causes one copy of each type of gene segment to go in any given lymphocyte, generating an enormous antibody repertoire.

Without wishing to be bound by a particular theory, V(D)J recombination in mammals rearranges variable (V), joining (J), and in some cases, diversity (D) gene segments in a nearly random fashion, in the primary lymphoid organs (bone marrow for B cells and thymus for T cells). For instance, the heavy chain is produced in a developing B cell via V(D)J recombination: (1) the first recombination event to occur is between one D and one J gene segment of the heavy chain locus so that any DNA between these two gene segments is deleted; and followed by (2) the joining of one V gene segment, from a region upstream of the newly formed DJ complex, forming a rearranged VDJ gene segment, so that all other gene segments between V and D segments are now deleted from the cell's genome. Primary transcript (unspliced RNA) is generated containing the VDJ region of the heavy chain and both the constant mu and delta chains (Cμ and Cδ) (i.e. the primary transcript contains the segments: V-D-J-Cμ-Cδ). The primary RNA is processed to add a polyadenylated (poly-A) tail after the Cμ chain and to remove sequence between the VDJ segment and this constant gene segment. Translation of this mRNA leads to the production of the IgM heavy chain protein. Similarly, the immunoglobulin light chain loci—the kappa (κ) and lambda (λ) chains—each rearrange this way, except that the light chains lack a D segment. In other words, the first step of recombination for the light chains involves the joining of the V and J chains to give a VJ complex before the addition of the constant chain gene during primary transcription. Translation of the spliced mRNA for either the kappa or lambda chains results in formation of the Ig κ or Ig λ light chain protein. Assembly of the Ig μ heavy chain and one of the light chains results in the formation of membrane bound form of the immunoglobulin IgM that is expressed on the surface of the immature B cell.

The variable domains can then be subjected to SHM to allow affinity maturation for a particular antigen. Without wishing to be bound by a particular theory, a general mechanism of SHM is as follows. SHM involves deamination of cytosine to uracil in DNA by the enzyme activation-induced cytidine deaminase, or AID. A cytosine:guanine pair is thus directly mutated to a uracil:guanine mismatch (FIG. 1). Uracil residues are not normally found in DNA, therefore most of these mutations—the uracil bases—are removed by uracil-DNA glycosylase, a high-fidelity Base excision repair enzyme, to maintain the integrity of the genome. Error-prone DNA polymerases are then recruited to fill in the gap and create mutations. Error-prone DNA polymerases often introduce mutations at the position of the deaminated cytosine itself or neighboring base pairs. One model of SHM is the DNA deamination model, which is based on AID and short-patch error-prone DNA repair by DNA polymerase-eta operating around AID C-to-U lesions. A key feature is its critical dependence on the gap-filling error prone DNA repair synthesis properties of DNA polymerase-eta targeting A:T base pairs at AID-mediated C-to-U lesions or ssDNA nicks. Another model of SHM is the reverse transcriptase model, which involves error-prone cDNA synthesis via an RNA-dependent DNA polymerase copying the base modified Ig pre-mRNA template and integrating the now error-filled cDNA copy back into the normal chromosomal site. This mechanism critically depends on AID C-to-U DNA lesions and long tract error-prone cDNA synthesis of the transcribed strand by DNA polymerase-eta acting as a reverse transcriptase.

During B cell division, the immunoglobulin variable region DNA is transcribed and translated. The introduction of mutations in the rapidly proliferating population of B cells ultimately culminates in the production of thousands of B cells, possessing slightly different receptors and varying specificity for the antigen, from which the B cell with highest affinities for the antigen can be selected. “Affinity maturation” is the process by which T_{FH} cell-activated B cells produce antibodies with increased affinity for antigen during the course of an immune response. It primarily occurs on surface immunoglobulin of germinal center B cells and as a direct result of somatic hypermutation (SHM) and selection by T_{FH} cells. With repeated exposures to the same antigen, a host will produce antibodies of successively greater affinities (e.g., a secondary response can elicit antibodies with several fold greater affinity than in a primary response). The B cells with the greatest affinity will then be selected to differentiate into plasma cells producing antibody and long-lived memory B cells contributing to enhanced immune responses upon reinfection.

1. An in vitro method, comprising:
providing a library of phage or phagemid clones;
diversifying the library of phage or phagemid clones by contacting the library of phage or phagemid clones with activation-induced deoxycytidine deaminase (AID);
further diversifying the library of phage or phagemid clones by contacting the library of phage or phagemid clones with DNA polymerase eta (Pol η); and
transfecting the diversified library into bacteria; and
generating a phage library.

2. The method of claim 1, further comprising:
panning the phage library against an antigen.

3. The method of claim 2, wherein the diversifying, the further diversifying, the transfecting, the generating, and the panning are repeated one or more times.

4. The method of claim 1, wherein the library of phage clones is made by a method, comprising:
providing a library of naïve IgV genes;
diversifying the library of naïve IgV genes by contacting the library of naïve IgV genes with activation-induced deoxycytidine deaminase (AID);
further diversifying the library of naïve IgV genes by contacting the library of naïve IgV genes with DNA polymerase eta (Pol η); and
generating a phage library;
optionally further comprising panning the phage library against at least one antigen to generate the library of phage clones.

5. The method of claim 1, further comprising producing the library of phage or phagemid clones by:
providing a library of naïve IgV genes;
diversifying the library of naïve IgV genes by contacting the library of naïve IgV genes with activation-induced deoxycytidine deaminase (AID);
further diversifying the library of naïve IgV genes by contacting the library of naïve IgV genes with DNA polymerase eta (Pol η), thereby generating a library of diversified IgV genes; and
generating a phage or phagemid library by cloning the library of diversified IgV genes into a quantity of phage or phagemid vectors; and
panning the phage or phagemid library against at least one antigen to produce the library of phage or phagemid clones.

6. The method of claim 1, wherein diversifying the library of phage or phagemid clones, further diversifying the library of phage or phagemid clones, or both, comprises formation of a single stranded DNA cassette to generate gapped DNA.

7. The method of claim 4, wherein the library of naïve IgV genes is provided in a quantity of gapped, double stranded DNA vectors, wherein each of said gapped, double stranded DNA vectors has a segment which exposes one or more of the naïve IgV genes in a single-stranded form, thereby containing a gap in the other strand of the double stranded DNA vectors.

8. The method of claim 2, wherein the antigen is glucagon-like peptide-1 receptor (GLP1R), AID, artemin, or fatty acid amide hydrolase (FAAH).

9. A method of creating a repertoire of mutagenized immunoglobulin variable (IgV) genes or diversifying a repertoire of IgV genes, comprising:
providing a quantity of polynucleotides comprising the IgV genes,
inducing mutagenesis in the IgV genes by incubating the quantity of polynucleotides with activation-induced deoxycytidine deaminase (AID) and amplifying the quantity of polynucleotides in the presence of polymerase eta (Pol η),
whereby the mutagenesis in the IgV genes creates the repertoire of mutagenized IgV genes or diversifying the repertoire of the IgV genes.

10. The method of claim 9, wherein IgV genes in the quantity of polynucleotides comprise an immunoglobulin heavy chain variable region gene (VH) segment and an immunoglobulin light chain variable region gene (VL) segment, or the quantity of polynucleotides each comprises a single-chain variable fragment (scFv).

11. The method of claim 9, wherein the quantity of polynucleotides comprises a library of llama VHH antibody fragments.

12. The method of claim 9, wherein the quantity of polynucleotides is in the form of gapped, double-stranded vectors, and providing the quantity of polynucleotides comprises:
denaturing a first double-stranded vector without the IgV genes and a second double-stranded vector with the IgV genes to form a mixture, wherein at least the first double-stranded vector without the IgV genes is cleaved or nicked to result in a gap, and
cooling the mixture in order to generate re-annealed vectors, wherein at least one of the re-annealed vectors comprises a first strand containing the gap derived from the first double-stranded vector without the IgV genes and a second strand with the IgV genes derived from the second double-stranded vector, and wherein the gap in the first strand exposes the IgV genes in the second strand as a single-stranded segment in the at least one of the re-annealed vectors.

13. The method of claim 9, wherein the quantity of polynucleotides further comprises one or more restriction enzyme sites for recognition by one or more restriction enzymes.

14. A method of providing a library of phage or phagemid clones with mutagenized IgV genes, comprising:
creating a repertoire of IgV genes according to a method of claim 9, and
cloning the repertoire of mutagenized IgV genes into phage vectors or phagemid vectors, thereby providing the library of phage or phagemid clones with mutagenized IgV genes.

15. The method of claim 14, further comprising diversifying the mutagenized IgV in the phage or phagemid clones by contacting the phage or phagemid clones with AID and Pol η.

16. A method of providing a phage or phagemid display library with mutagenized IgV genes, comprising:
providing a library of phage or phagemid clones with mutagenized IgV genes according to the method of claim 14, and
introducing the library of phage or phagemid clones into a quantity of bacteria, thereby providing a phage or phagemid display library with mutagenized IgV genes.

17. The method of claim 16, further comprising titrating the phage or phagemid display library to measure transducing unit of the phage or phagemid display library.

18. A method of producing, screening for, and/or isolating an antibody or an antigen-binding fragment thereof, comprising:
providing a phage or phagemid display library with mutagenized IgV genes according to a method of claim 15, and
panning the phage or phagemid display library against an antigen to detect a bound phage or phagemid clone to the antigen,
whereby IgV genes in the bound phage or phagemid clone encodes the antibody or fragment that binds the antigen.

19. The method of claim 18, wherein the panning comprises two or more rounds of binding assays independently selected from chromatographic column, enzyme-linked immunosorbent assay (ELISA), surface plasmon resonance (SPR).

20. A phage or phagemid library made by the method of claim 1.

21. A library of phage or phagemid clones made by the method of claim 14.

22. A repertoire or library of polynucleotides comprising mutagenized IgV genes, which is made by a method of claim 9.

23. An antibody, a single-chain variable fragment (scFv), a single domain antibody, or a nanobody-based human heavy chain antibody, made by the method of claim 2.

24. The antibody, the scFv, the single domain antibody, or the nanobody-based human heavy chain antibody of claim 23, which binds human glucagon-like peptide-1 receptor (GLP1R),
wherein the antibody is an anti-human GLP1R antibody, comprising:
a variable heavy (VH) chain having a peptide sequence of SEQ ID NO:112 or encoded by a DNA sequence of SEQ ID NO:111, and
a variable light (VL) chain having a peptide sequence of SEQ ID NO: 114 or encoded by a DNA sequence of SEQ ID NO:113;
wherein the scFv which binds human GLP1R comprises:
a VH chain having a peptide sequence of SEQ ID NO:112 or encoded by a DNA sequence of SEQ ID NO:111, and
a VL chain having a peptide sequence of SEQ ID NO: 114 or encoded by a DNA sequence of SEQ ID NO:113; or
wherein the single domain antibody which binds human GLP1R comprises a VH chain having a peptide sequence of SEQ ID NO:112 or encoded by a DNA sequence of SEQ ID NO:111.

25. The antibody, the scFv, the single domain antibody, or the nanobody-based human heavy chain antibody of claim 23, which binds human activation-induced deoxycytidine deaminase (AID),
wherein the antibody which binds human AID is an anti-human AID antibody, comprising:
a VH chain having a peptide sequence of SEQ ID NO:120 or encoded by a DNA sequence of SEQ ID NO:119, and
a VL chain having a peptide sequence of SEQ ID NO: 122 or encoded by a DNA sequence of SEQ ID NO:121;
wherein the scFv which binds human AID comprises:
a VH chain having a peptide sequence of SEQ ID NO:120 or encoded by a DNA sequence of SEQ ID NO:119, and
a VL chain having a peptide sequence of SEQ ID NO: 122 or encoded by a DNA sequence of SEQ ID NO:121; or
wherein the single domain antibody which binds human AID comprises a VH chain having a peptide sequence of SEQ ID NO:120 or encoded by a DNA sequence of SEQ ID NO:119.

26. The antibody, the scFv, the single domain antibody, or the nanobody-based human heavy chain antibody of claim 23, being an anti-artemin single domain antibody, comprising a variable domain of llama heavy chain antibody (VHH) having a peptide sequence of SEQ ID NO:118 or encoded by a DNA sequence of SEQ ID NO:117.

27. The antibody, the scFv, the single domain antibody, or the nanobody-based human heavy chain antibody of claim 23, being an anti-fatty acid amide hydrolase single domain antibody, comprising a VHH having a peptide sequence of SEQ ID NO:116 or encoded by a DNA sequence of SEQ ID NO:115.

28. A kit, comprising:
(I) a quantity of naïve IgV genes;
a quantity of activation-induced deoxycytidine deaminase (AID);
a quantity of DNA polymerase eta (Pol η); and
instructions for use,
or
(II) a library of phage or phagemid clones;
a quantity of activation-induced deoxycytidine deaminase (AID),
a quantity of DNA polymerase eta (Pol η); and
instructions for use.

29. (canceled)


SEQUENCE LISTING (122 sequences)

SEQ ID NO: 1 - protein, 20 aa, Artificial Sequence
  synthetic construct
GGGGSGGGGSGGGGSGGGGS

SEQ ID NO: 2 - DNA, 45 bp, Artificial Sequence
  synthetic construct
ggtggtggtggttctggtggtggtggttctggcggcggcggctcc

SEQ ID NO: 3 - DNA, 30 bp, Artificial Sequence
  synthetic construct
ggcggcggcggctccggtggtggtggatcc

SEQ ID NO: 4 - DNA, 21 bp, Artificial Sequence
  synthetic construct
tactcgcggcccagccggcca

SEQ ID NO: 5 - DNA, 21 bp, Artificial Sequence
  synthetic construct
tggtgttggcctcccgggcca

SEQ ID NO: 6 - DNA, 21 bp, Artificial Sequence
  synthetic construct
tactcgcggcccacgcggcca

SEQ ID NO: 7 - DNA, 22 bp, Artificial Sequence
  synthetic construct
tggtgttggcctcagcggcact

SEQ ID NO: 8 - DNA, 15 bp, Artificial Sequence
  synthetic construct
ggcggcggcggctcc

SEQ ID NO: 9 - DNA, 29 bp, Artificial Sequence
  synthetic construct
tattactcgcggcccacgcggccatggct

SEQ ID NO: 10 - DNA, 39 bp, Artificial Sequence
  synthetic construct
ggtgatggtgttggccccaggggctgaggagacggtgac

SEQ ID NO: 11 - DNA, 21 bp, Artificial Sequence
  synthetic construct
tttggagattttcaacgttga

SEQ ID NO: 12 - DNA, 24 bp, Artificial Sequence
  synthetic construct
gcgtaacgatctaaagttttgtcg

SEQ ID NO: 13 - protein, 6 aa, Artificial Sequence
  HHHHHH
HHHHHH

SEQ ID NO: 14 - protein, 19 aa, Unknown
  f3TR1-scFv phage display vector
VRHRGKLRDGDNFEAPGDF

SEQ ID NO: 15 - protein, 11 aa, Unknown
  f3TR1-scFv phage display vector
AGWDDSLSAWV

SEQ ID NO: 16 - protein, 12 aa, Unknown
  f3TR1-scFv phage display vector
AGRYSGSSGLDY

SEQ ID NO: 17 - protein, 11 aa, Unknown
  f3TR1-scFv phage display vector
ASWDDSLDAAI

SEQ ID NO: 18 - protein, 11 aa, Unknown
  f3TR1-scFv phage display vector
VRDSGLSGVDY

SEQ ID NO: 19 - protein, 9 aa, Unknown
  f3TR1-scFv phage display vector
QQNYKSTWT

SEQ ID NO: 20 - protein, 15 aa, Unknown
  f3TR1-scFv phage display vector
ARDPSGCYYGGTFDY

SEQ ID NO: 21 - protein, 9 aa, Unknown
  f3TR1-scFv phage display vector
LQRDTFPYT

SEQ ID NO: 22 - protein, 13 aa, Unknown
  f3TR1-scFv phage display vector
ARSRSSGLYHFDL

SEQ ID NO: 23 - protein, 9 aa, Unknown
  f3TR1-scFv phage display vector
HSFDSGSQV

SEQ ID NO: 24 - protein, 11 aa, Unknown
  f3TR1-scFv phage display vector
TDPNWTELVRP

SEQ ID NO: 25 - protein, 11 aa, Unknown
  f3TR1-scFv phage display vector
QVWDSGSNHMV

SEQ ID NO: 26 - protein, 11 aa, Unknown
  f3TR1-scFv phage display vector
ARPGYSYGYDY

SEQ ID NO: 27 - protein, 9 aa, Unknown
  f3TR1-scFv phage display vector
QSSDSTNWV

SEQ ID NO: 28 - protein, 17 aa, Unknown
  f3TR1-scFv phage display vector
ARTNGFDFWSGPPSVDV

SEQ ID NO: 29 - protein, 11 aa, Unknown
  f3TR1-scFv phage display vector
SSRDTRGKHLV

SEQ ID NO: 30 - protein, 13 aa, Unknown
  f3TR1-scFv phage display vector
ARYLDSSANFLGY

SEQ ID NO: 31 - protein, 9 aa, Unknown
  f3TR1-scFv phage display vector
MQNLQTPFT

SEQ ID NO: 32 - protein, 13 aa, Unknown
  f3TR1-scFv phage display vector
AIGRDGYNLDFDY

SEQ ID NO: 33 - protein, 9 aa, Unknown
  f3TR1-scFv phage display vector
QQSYSTPWT

SEQ ID NO: 34 - protein, 9 aa, Unknown
  f3TR1-scFv phage display vector
ARGTTHFDY

SEQ ID NO: 35 - protein, 10 aa, Unknown
  f3TR1-scFv phage display vector
QSYDSSNPWV

SEQ ID NO: 36 - protein, 15 aa, Unknown
  f3TR1-scFv phage display vector
AHRLGMPRGTPYFDY

SEQ ID NO: 37 - protein, 9 aa, Unknown
  f3TR1-scFv phage display vector
QQYGHFPRT

SEQ ID NO: 38 - protein, 12 aa, Unknown
  f3TR1-scFv phage display vector
VRFRPQTTTGDY

SEQ ID NO: 39 - protein, 11 aa, Unknown
  f3TR1-scFv phage display vector
ATWDDSVNGPG

SEQ ID NO: 40 - protein, 14 aa, Unknown
  f3TR1-scFv phage display vector
ARQSGTQTEVTFDI

SEQ ID NO: 41 - protein, 9 aa, Unknown
  f3TR1-scFv phage display vector
QKYDGAPRT

SEQ ID NO: 42 - protein, 12 aa, Unknown
  f3TR1-scFv phage display vector
AATYNWNDVGDY

SEQ ID NO: 43 - protein, 10 aa, Unknown
  f3TR1-scFv phage display vector
LQHDNFPLGP

SEQ ID NO: 44 - protein, 13 aa, Unknown
  f3TR1-scFv phage display vector
AKGIRGSTRSHDY

SEQ ID NO: 45 - protein, 11 aa, Unknown
  f3TR1-scFv phage display vector
NSRDSSGNHVV

SEQ ID NO: 46 - DNA, 23 bp, Artificial Sequence
  synthetic construct
caggtbcagctggtgcagtctgg

SEQ ID NO: 47 - DNA, 23 bp, Artificial Sequence
  synthetic construct
carrtscagctggtrcartctgg

SEQ ID NO: 48 - DNA, 23 bp, Artificial Sequence
  synthetic construct
cagrtcaccttgaaggagtctgg

SEQ ID NO: 49 - DNA, 23 bp, Artificial Sequence
  synthetic construct
sargtgcagctggtggagtctgg

SEQ ID NO: 50 - DNA, 23 bp, Artificial Sequence
  synthetic construct
gaggtgcagctgktggagwcysg

SEQ ID NO: 51 - DNA, 23 bp, Artificial Sequence
  synthetic construct
caggtgcarctgcaggagtcggg

SEQ ID NO: 52 - DNA, 23 bp, Artificial Sequence
  synthetic construct
cagstgcagctrcagsagtssgg

SEQ ID NO: 53 - DNA, 23 bp, Artificial Sequence
  synthetic construct
gargtgcagctggtgcagtctgg

SEQ ID NO: 54 - DNA, 23 bp, Artificial Sequence
  synthetic construct
caggtacagctgcagcagtcagg

SEQ ID NO: 55 - DNA, 21 bp, Artificial Sequence
  synthetic construct
aagggttggggcggatgcact

SEQ ID NO: 56 - DNA, 21 bp, Artificial Sequence
  synthetic construct
gaccgatgggcccttggtgga

SEQ ID NO: 57 - DNA, 23 bp, Artificial Sequence
  synthetic construct
gacatccagatgacccagtctcc

SEQ ID NO: 58 - DNA, 23 bp, Artificial Sequence
  synthetic construct
gmcatccrgwtgacccagtctcc

SEQ ID NO: 59 - DNA, 23 bp, Artificial Sequence
  synthetic construct
gatrttgtgatgacycagwctcc

SEQ ID NO: 60 - DNA, 23 bp, Artificial Sequence
  synthetic construct
gaaatwgtgwtgacrcagtctcc

SEQ ID NO: 61 - DNA, 23 bp, Artificial Sequence
  synthetic construct
gacatcgtgatgacccagtctcc

SEQ ID NO: 62 - DNA, 23 bp, Artificial Sequence
  synthetic construct
gaaacgacactcacgcagtctcc

SEQ ID NO: 63 - DNA, 23 bp, Artificial Sequence
  synthetic construct
gawrttgtgmtgacwcagtctcc

SEQ ID NO: 64 - DNA, 24 bp, Artificial Sequence
  synthetic construct
acactctcccctgttgaagctctt

SEQ ID NO: 65 - DNA, 23 bp, Artificial Sequence
  synthetic construct
cagtctgtgctgactcagccacc

SEQ ID NO: 66 - DNA, 23 bp, Artificial Sequence
  synthetic construct
cagtctgtgytgacgcagccgcc

SEQ ID NO: 67 - DNA, 21 bp, Artificial Sequence
  synthetic construct
cagtctgccctgactcagcct

SEQ ID NO: 68 - DNA, 23 bp, Artificial Sequence
  synthetic construct
tcctatgwgctgacwcagccacc

SEQ ID NO: 69 - DNA, 23 bp, Artificial Sequence
  synthetic construct
tcttctgagctgactcaggaccc

SEQ ID NO: 70 - DNA, 21 bp, Artificial Sequence
  synthetic construct
ctgcctgtgctgactcagccc

SEQ ID NO: 71 - DNA, 23 bp, Artificial Sequence
  synthetic construct
cagcytgtgctgactcaatcryc

SEQ ID NO: 72 - DNA, 20 bp, Artificial Sequence
  synthetic construct
cagsctgtgctgactcagcc

SEQ ID NO: 73 - DNA, 23 bp, Artificial Sequence
  synthetic construct
aattttatgctgactcagcccca

SEQ ID NO: 74 - DNA, 23 bp, Artificial Sequence
  synthetic construct
cagrctgtggtgacycaggagcc

SEQ ID NO: 75 - DNA, 23 bp, Artificial Sequence
  synthetic construct
cagscwgkgctgactcagccacc

SEQ ID NO: 76 - DNA, 23 bp, Artificial Sequence
  synthetic construct
tgaacattctgtaggggccactg

SEQ ID NO: 77 - DNA, 23 bp, Artificial Sequence
  synthetic construct
tgaacattccgtaggggcaactg

SEQ ID NO: 78 - DNA, 49 bp, Artificial Sequence
  synthetic construct
tactcgcggcccacgcggccatggctcaggtbcagctggtgcagtctgg

SEQ ID NO: 79 - DNA, 49 bp, Artificial Sequence
  synthetic construct
tactcgcggcccacgcggccatggctcarrtscagctggtrcartctgg

SEQ ID NO: 80 - DNA, 49 bp, Artificial Sequence
  synthetic construct
tactcgcggcccacgcggccatggctcagrtcaccttgaaggagtctgg

SEQ ID NO: 81 - DNA, 49 bp, Artificial Sequence
  synthetic construct
tactcgcggcccacgcggccatggctsargtgcagctggtggagtctgg

SEQ ID NO: 82 - DNA, 49 bp, Artificial Sequence
  synthetic construct
tactcgcggcccacgcggccatggctgaggtgcagctgktggagwcysg

SEQ ID NO: 83 - DNA, 49 bp, Artificial Sequence
  synthetic construct
tactcgcggcccacgcggccatggctcaggtgcarctgcaggagtcggg

SEQ ID NO: 84 - DNA, 49 bp, Artificial Sequence
  synthetic construct
tactcgcggcccacgcggccatggctcagstgcagctrcagsagtssgg

SEQ ID NO: 85 - DNA, 49 bp, Artificial Sequence
  synthetic construct
tactcgcggcccacgcggccatggctgargtgcagctggtgcagtctgg

SEQ ID NO: 86 - DNA, 49 bp, Artificial Sequence
  synthetic construct
tactcgcggcccacgcggccatggctcaggtacagctgcagcagtcagg

SEQ ID NO: 87 - DNA, 63 bp, Artificial Sequence
  synthetic construct
ggagccgccgccgccagaaccaccaccaccagaaccaccaccaccggttggggcggatgcact

SEQ ID NO: 88 - DNA, 66 bp, Artificial Sequence
  synthetic construct
ggagccgccgccgccagaaccaccaccaccagaaccaccaccaccgaccgatgggcccttggtgga

SEQ ID NO: 89 - DNA, 53 bp, Artificial Sequence
  synthetic construct
ggcggcggcggctccggtggtggtggatccgacatccagatgacccagtctcc

SEQ ID NO: 90 - DNA, 53 bp, Artificial Sequence
  synthetic construct
ggcggcggcggctccggtggtggtggatccgmcatccrgwtgacccagtctcc

SEQ ID NO: 91 - DNA, 53 bp, Artificial Sequence
  synthetic construct
ggcggcggcggctccggtggtggtggatccgatrttgtgatgacycagwctcc

SEQ ID NO: 92 - DNA, 53 bp, Artificial Sequence
  synthetic construct
ggcggcggcggctccggtggtggtggatccgaaatwgtgwtgacrcagtctcc

SEQ ID NO: 93 - DNA, 53 bp, Artificial Sequence
  synthetic construct
ggcggcggcggctccggtggtggtggatccgacatcgtgatgacccagtctcc

SEQ ID NO: 94 - DNA, 53 bp, Artificial Sequence
  synthetic construct
ggcggcggcggctccggtggtggtggatccgaaacgacactcacgcagtctcc

SEQ ID NO: 95 - DNA, 53 bp, Artificial Sequence
  synthetic construct
ggcggcggcggctccggtggtggtggatccgawrttgtgmtgacwcagtctcc

SEQ ID NO: 96 - DNA, 49 bp, Artificial Sequence
  synthetic construct
tggtgttggcctcagcggcactagtgaagacagatggtgcagccacagt

SEQ ID NO: 97 - DNA, 53 bp, Artificial Sequence
  synthetic construct
ggcggcggcggctccggtggtggtggatcccagtctgtgctgactcagccacc

SEQ ID NO: 98 - DNA, 53 bp, Artificial Sequence
  synthetic construct
ggcggcggcggctccggtggtggtggatcccagtctgtgytgacgcagccgcc

SEQ ID NO: 99 - DNA, 51 bp, Artificial Sequence
  synthetic construct
ggcggcggcggctccggtggtggtggatcccagtctgccctgactcagcct

SEQ ID NO: 100 - DNA, 53 bp, Artificial Sequence
  synthetic construct
ggcggcggcggctccggtggtggtggatcctcctatgwgctgacwcagccacc

SEQ ID NO: 101 - DNA, 53 bp, Artificial Sequence
  synthetic construct
ggcggcggcggctccggtggtggtggatcctcttctgagctgactcaggaccc

SEQ ID NO: 102 - DNA, 51 bp, Artificial Sequence
  synthetic construct
ggcggcggcggctccggtggtggtggatccctgcctgtgctgactcagccc

SEQ ID NO: 103 - DNA, 53 bp, Artificial Sequence
  synthetic construct
ggcggcggcggctccggtggtggtggatcccagcytgtgctgactcaatcryc

SEQ ID NO: 104 - DNA, 50 bp, Artificial Sequence
  synthetic construct
ggcggcggcggctccggtggtggtggatcccagsctgtgctgactcagcc

SEQ ID NO: 105 - DNA, 53 bp, Artificial Sequence
  synthetic construct
ggcggcggcggctccggtggtggtggatccaattttatgctgactcagcccca

SEQ ID NO: 106 - DNA, 53 bp, Artificial Sequence
  synthetic construct
ggcggcggcggctccggtggtggtggatcccagrctgtggtgacycaggagcc

SEQ ID NO: 107 - DNA, 53 bp, Artificial Sequence
  synthetic construct
ggcggcggcggctccggtggtggtggatcccagscwgkgctgactcagccacc

SEQ ID NO: 108 - DNA, 49 bp, Artificial Sequence
  synthetic construct
tggtgttggcctcagcggcactagtagaggasggygggaacagagtgac

SEQ ID NO: 109 - DNA, 360 bp, Unknown
  f3TR1-scFv phage display vector
caggtacagctgcagcagtcaggtccaggactggtgaagccttcgcagaccttctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattattgtgtaagagatagtggtttgtcgggggtggactactggggccagggaaccctggtcaccgtctcc

SEQ ID NO: 110 - DNA, 313 bp, Unknown
  f3TR1-scFv phage display vector
gacatccagatgacccagtctccattgtccctgtctacatctgtcggagacagagccaccatcacctgtcgggcgagtcaagatatcagcgtctatgtaaattggtatcaacaacagcccgggaaagcccctagactctcgatctatgcttcctcccatttacagaccggtgtcccgtcaacattcagtggccgtggatctgggacagatttcactctcaccatcagcagtctccaacttgacgattttactacttattattgtcaacaaaactacaagtccacgtggacgttcggccaggggaccaaggtgg

SEQ ID NO: 111 - DNA, 357 bp, Unknown
  f3TR1-scFv phage display vector
gtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgaggggtcgggatgattactatgatagtagtgcctttgactactggggccagggaaccctggtcaccgtc

SEQ ID NO: 112 - protein, 119 aa, Unknown
  f3TR1-scFv phage clone
VQLVQSGAEVKKPGSSVKVSCKASGGTFSSYAISWVRQAPGQGLEWMGGIIPIFGTANYAQKFQGRVTITADESTSTAYMELSSLRSEDTAVYYCARGRDDYYDSSAFDYWGQGTLVTV

SEQ ID NO: 113 - DNA, 318 bp, Unknown
  f3TR1-scFv phage display vector
gtgctgactcagccaccctcggtgtcagtggccccaggacagacggccagggttacctgtgggggaaacaagattggaagttacagtgtctactggtaccagcacaagcctggccaggcccctgtgttggtcgtctatgatgatagtgaccggccctcagggatccctgaacgattctctggctccaactctgggaacatggccaccctgaccatcagcggggtcgaagccggggatgaggccgactattactgtcaggtgtgggattgtactactgatgatgtgatcttcggcggagggaccaagctgaccgtccta

SEQ ID NO: 114 - protein, 106 aa, Unknown
  f3TR1-scFv phage clone
VLTQPPSVSVAPGQTARVTCGGNKIGSYSVYWYQHKPGQAPVLVVYDDSDRPSGIPERFSGSNSGNMATLTISGVEAGDEADYYCQVWDCTTDDVIFGGGTKLTVL

SEQ ID NO: 115 - DNA, 324 bp, Unknown
  f3TR1-scFv phage display vector
gtgcagcttgtggagtctgggggaggcttggtgcaggctggggggtctctgagactctcctgtgcagcctctggacgaaccttcagtgacaacaccatggcctggcaccgccagggtccagggaagcagcgcgggttggtcgcatttatcacaactggtggccgtacagtctataccgactccgtgaagggccgattcatcatctccagagacaacgccaagaacacggggtttctgcaaatgaacagcctgaaacctgaggacacggccgtctattactgttatgtagccggtaattggggccaggggacccaggtcaccgtc

SEQ ID NO: 116 - protein, 108 aa, Unknown
  f3TR1-scFv phage clone
VQLVESGGGLVQAGGSLRLSCAASGRTFSDNTMAWHRQGPGKQRGLVAFITTGGRTVYTDSVKGRFIISRDNAKNTGFLQMNSLKPEDTAVYYCYVAGNWGQGTQVTV

SEQ ID NO: 117 - DNA, 375 bp, Unknown
  f3TR1-scFv phage display vector
gtgcagctggtagagtctgggggaggattggtgcaggctggggcctctctgagactctcctgtgcagcctctggaggtactctcgtcaactctagtatgggctggttccgccaggctccagggaaggagcgtgacttcgtggcaactattaactggcgcggtgatactacatggtatggagagtccgtgaagggccgattctccatctccagagacaacaccaaaaacatggtgtacctgcaaatgaacagcctgaaacctgaggacacggccgtttattactgtgcagtctacaggacaagatactatactggccgtcgcatgatgtccccagatgagtatgacgtttggggccaggggacccaggtcaccgtc

SEQ ID NO: 118 - protein, 125 aa, Unknown
  f3TR1-scFv phage clone
VQLVESGGGLVQAGASLRLSCAASGGTLVNSSMGWFRQAPGKERDFVATINWRGDTTWYGESVKGRFSISRDNTKNMVYLQMNSLKPEDTAVYYCAVYRTRYYTGRRMMSPDEYDVWGQGTQVTV

SEQ ID NO: 119 - DNA, 363 bp, Unknown
  f3TR1-scFv phage display vector
gtgcagctggtgcagtctgggggaggcttggtacagcctggggggtctctgcgactctcctgtgtagcctctggattcacctttaccgactatgccgtgacctgggtccgccaggctccagggaagggtctggagtgggtctcagctatcagtggtagtggtggaaacacatactacgcagactccgtgaagggccggttcaccgtctccagggacaattccgacaacacggtgtctctgcaaatgaacagcctgagagtcgaggacacgggcatatattattgtgcgaagtcggctcggatcttaaatggatattacttctatgctatggacgtctggggccaagggaccacggtcaccgtc

SEQ ID NO: 120 - protein, 121 aa, Unknown
  f3TR1-scFv phage clone
VQLVQSGGGLVQPGGSLRLSCVASGFTFTDYAVTWVRQAPGKGLEWVSAISGSGGNTYYADSVKGRFTVSRDNSDNTVSLQMNSLRVEDTGIYYCAKSARILNGYYFYAMDVWGQGTTVTV

SEQ ID NO: 121 - DNA, 330 bp, Unknown
  f3TR1-scFv phage display vector
cagtctgccctgactcagcctgcctccgtgtctgggtctcctggacagtcgatcaccgtctcctgcactggaaccagcagtgatgttgcgaaatataaccttgtctcctggtaccaacagcacccaggcaaagcccccgaactcatgatttatgatgtcagtaagcggccctcaggggtccctgatcgcttctctggctccaagtctggcaacacggcctccctgaccatttctgggctccagcctgacgatgaggcttattatcactgcagctcatatggaggcaccaacaatttgctattcggcggagggaccaaggtgaccgttcta

SEQ ID NO: 122 - protein, 110 aa, Unknown
  f3TR1-scFv phage clone
QSALTQPASVSGSPGQSITVSCTGTSSDVAKYNLVSWYQQHPGKAPELMIYDVSKRPSGVPDRFSGSKSGNTASLTISGLQPDDEAYYHCSSYGGTNNLLFGGGTKVTVL